(19) United States
(12) Patent Application Publication
(10) Pub. No.: US 2023/0157319 A1
(43) Pub. Date: May 25, 2023

(54) SUPPLEMENTS AND COMPOSITIONS CONTAINING AMINO ACIDS AND IGF-1 AND METHODS OF USE

(71) Applicant: PURETEIN BIOSCIENCE LLC, St. Louis Park, MN (US)

(72) Inventors: Brett Casebolt, St. Louis Park, MN (US); Mathew A. Vaughn, St. Louis Park, MN (US)

(73) Assignee: PURETEIN BIOSCIENCE LLC, St. Louis Park, MN (US)

(21) Appl. No.: 17/917,506

(22) PCT Filed: Apr. 7, 2021
(86) PCT No.: PCT/US2021/026157

§ 371 Date: Oct. 6, 2022

Related U.S. Application Data
(60) Provisional application No. 63/006,313, filed on Apr. 7, 2020.

(51) Int. Cl. A23K 20/142; A23K 20/184; A23K 50/30; A23K 50/75

(52) U.S. Cl. CPC A23K 20/142; A23K 20/184; A23K 50/30; A23K 50/75

(57) ABSTRACT

Provided herein are supplements that include free amino acids L-glutamine or a salt thereof, L-leucine or a salt thereof, nd L-arginine or a salt thereof, and active IGF-1. The L-glutamine or salt thereof, L-leucine or salt thereof, and L-arginine or salt thereof can be present at a ratio of approximately 3:1:1. Also provided are compositions that include the supplement, such as a food product. The disclosure also includes, but is not limited to, methods for using the supplements, including improving the performance of an animal, improving a processing factor of an animal, or increasing intestinal health of an animal, where the method includes administering to the animal a food product that has the supplement.

### FIELD

This disclosure relates generally to supplements and compositions having IGF-1 and specific amino acids and to methods of using the supplements and compositions.

### SUMMARY OF THE APPLICATION

Nutrition of animals early in life is important for development of the immune system and skeletal musculature system. Typically, feeds are provided ad libitum include an excess of components including grains, proteins, vitamins, minerals, and the like. The inventors have found that adding a combination of active IGF-1 and three amino acids to food results in an unexpected change in certain useful characteristics. The combination of IGF-1 and three amino acids results in a nutritional synergy, where the combination of IGF-1 and three amino acids is nutritionally superior to any of the components used alone at any tested dose. Moreover, the amount of the three amino acids is less than is generally used in animal food products.

Terms used herein will be understood to take on their ordinary meaning in the relevant art unless specified otherwise. Several terms used herein and their meanings are set forth below.

The terms “weight percent,” “weight %,” and “wt %” are used herein interchangeably and refer to the weight of a compound in a supplement or composition. For instance, 1 gram of an amino acid in 100 grams of a composition is 1 wt % of the amino acid in the composition.

The term “and/or” means one or all of the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Unless otherwise specified, “a,” “an,” “the,” and “at least one” are used interchangeably and mean one or more than one.

Conditions that are “suitable” for an event to occur, or “suitable” conditions, are conditions that do not prevent such events from occurring. Thus, these conditions permit, enhance, facilitate, and/or are conducive to the event.

As used herein, “substantially free of” a material refers to a supplement or composition having less than 10% of the material, less than 5% of the material, less than 4% of the material, less than 3% of the material, less than 2% of the material, or less than 1% of the material. In one embodiment, the presence of the material in a supplement or composition is undetectable.

As used herein, “providing” in the context of a supplement or composition, means making the supplement or composition, purchasing the composition or supplement, or otherwise obtaining the supplement or composition.

Also herein, the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, supplement, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, supplements, compositions, or characteristics may be combined in any suitable manner in one or more embodiments.

For any method disclosed herein that includes discrete steps, the steps may be conducted in any feasible order. And, as appropriate, any combination of two or more steps may be conducted simultaneously.

### BRIEF DESCRIPTION OF THE FIGURES

The following detailed description of illustrative embodiments of the present disclosure may be best understood when read in conjunction with the following drawings.

FIG. 1 shows porcine satellite cells treated with 1 of 8 treatments. The CON treatment consisted of low-glucose Dulbecco's Modified Eagle Medium supplemented with 2% (vol/vol) fetal bovine serum; IGF-1, CON media plus active 5 ng/mL IGF-1; GLUT, CON media plus 4 mM L-glutamine; LEU, CON media plus 1 mM L-leucine; ARG, CON media plus 1 mM L-arginine; LYS, CON media plus 10 mM L-lysine; MET, CON media plus 10 μL-methionine.^{a,b,c,d} Means with different superscripts differ P<0.05.

FIG. 2 shows porcine satellite cells treated with 1 of 3 treatments. The CON treatment consisted of low-glucose Dulbecco's Modified Eagle Medium supplemented with 2% (vol/vol) fetal bovine serum; IGF-1, CON media plus 5 ng/gml active IGF-1; COMBO, CON media plus 5 ng/mL IGF-1, 4 mM L-glutamine, 1 mM L-leucine, and 1 mM L-arginine.^{a,b,c} Means with different superscripts differ P<0.0001.

FIG. 3 shows porcine satellite cells treated with 1 of 7 treatments. The CON treatment consisted of low-glucose Dulbecco's Modified Eagle Medium supplemented with 2% (vol/vol) fetal bovine serum; IGF-1, CON media plus active 5 ng/mL IGF-1; COMBO 0, CON media plus 5 ng/mL active IGF-1, 0 mM L-glutamine, 1 mM L-leucine, and 1 mM L-arginine; COMBO 1, CON media plus active 5 ng/mL IGF-1, 1 mM L-glutamine, 1 mM L-leucine, and 1 mM L-arginine; COMBO 2, CON media plus active 5 ng/mL IGF-1, 2 mM L-glutamine, 1 mM L-leucine, and 1 mM L-arginine; COMBO 3, CON media plus active 5 ng/mL IGF-1, 3 mM L-glutamine, 1 mM L-leucine, and 1 mM L-arginine; COMBO 4, CON media plus active 5 ng/mL IGF-1, 4 mM L-glutamine, 1 mM L-leucine, and 1 mM L-arginine.^{a,b,c,d} Means with different superscripts differ P<0.05.

FIG. 4 shows porcine satellite cells were treated with 1 of 5 treatments. The CON treatment consisted of low-glucose Dulbecco's Modified Eagle Medium supplemented with 2% (vol/vol) fetal bovine serum; IGF-1, CON media plus 5 ng/g active IGF-1; COMBO 0.5, CON media plus 5 ng/mL IGF-1, 3 mM L-glutamine, 0.5 mM L-leucine, and 1 mM L-arginine; COMBO 1, CON media plus 5 ng/mL IGF-1, 3 mM L-glutamine, 1 mM L-leucine, and 1 mM L-arginine; COMBO 2, CON media plus 5 ng/mL IGF-1, 3 mM L-glutamine, 2 mM L-leucine, and 1 mM L-arginine.^{a,b,c,d,e} Means with different superscripts differ P<0.05.

FIG. 5 shows porcine satellite cells treated with 1 of 9 treatments. The CON treatment consisted of low-glucose Dulbecco's Modified Eagle Medium supplemented with 2% (vol/vol) fetal bovine serum; IGF-1, CON media plus 5 ng/mL active IGF-1; COMBO 0, CON media plus 5 ng/mL active IGF-1, 3 mM L-glutamine, 1 mM L-leucine, and 0 mM L-arginine; COMBO 0.1, CON media plus 5 ng/mL active IGF-1, 3 mM L-glutamine, 1 mM L-leucine, and 0.1 mM L-arginine; COMBO 0.25, CON media plus 5 ng/mL active IGF-1, 3 mM L-glutamine, 1 mM L-leucine, and 0.25 mM L-arginine; COMBO 0.5, CON media plus 5 ng/mL active IGF-1, 3 mM L-glutamine, 1 mM L-leucine, and 0.5 mM L-arginine; COMBO 1, CON media plus 5 ng/mL active IGF-1, 3 mM L-glutamine, 1 mM L-leucine, and 1 mM L-arginine; COMBO 2, CON media plus 5 ng/mL active IGF-1, 3 mM L-glutamine, 1 mM L-leucine, and 2 mM L-arginine; COMBO 3, CON media plus 5 ng/mL active IGF-1, 3 mM L-glutamine, 1 mM L-leucine, and 3 mM L-arginine.^{a,b,c,d,e,f} Means with different superscripts differ P<0.05.

FIG. 6 shows porcine satellite cells treated with 1 of 3 treatments. The CON treatment consisted of low-glucose Dulbecco's Modified Eagle Medium supplemented with 2% (vol/vol) fetal bovine serum; IGF-1, CON media plus 5 ng/mL active IGF-1; COMBO, CON media plus 5 ng/mL active IGF-1, 3 mM L-glutamine, 1 mM L-leucine, and 1 mM L-arginine; COMBO 0.5, CON media plus 5 ng/mL active IGF-1, 3 mM L-glutamine, 1 mM L-leucine, 1 mM L-arginine, and 0.5 mM L-methionine; COMBO 1, CON media plus 5 ng/mL active IGF-1, 3 mM L-glutamine, 1 mM L-leucine, 1 mM L-arginine, and 1 mM L-methionine; COMBO 2, CON media plus 5 ng/mL active IGF-1, 3 mM L-glutamine, 1 mM L-leucine, 1 mM L-arginine, and 2 mM L-methionine, COMBO 3; CON media plus 5 ng/mL active IGF-1, 3 mM L-glutamine, 1 mM L-leucine, 1 mM L-arginine, and 3 mM L-methionine.^{a,b,c,d,e} Means with different superscripts differ P<0.05.

FIG. 7 shows porcine satellite cells treated with 1 of 3 treatments. The CON treatment consisted of low-glucose Dulbecco's Modified Eagle Medium supplemented with 2% (vol/vol) fetal bovine serum; IGF-1, CON media plus 5 ng/mL active IGF-1; COMBO, CON media plus 5 ng/mL active IGF-1, 3 mM L-glutamine, 1 mM L-leucine, and 1 mM L-arginine; COMBO 0.5, CON media plus 5 ng/mL active IGF-1, 3 mM L-glutamine, 1 mM L-leucine, 1 mM L-arginine, and 0.5 mM L-lysine; COMBO 1, CON media plus 5 ng/mL active IGF-1, 3 mM L-glutamine, 1 mM L-leucine, 1 mM L-arginine, and 1 mM L-lysine; COMBO 2, CON media plus 5 ng/mL active IGF-1, 3 mM L-glutamine, 1 mM L-leucine, 1 mM L-arginine, and 2 mM L-lysine; COMBO 3, CON media plus 5 ng/mL active IGF-1, 3 mM L-glutamine, 1 mM L-leucine, 1 mM L-arginine, and 3 mM L-lysine.^{a,b,c,d} Means with different superscripts differ P<0.05.

FIG. 8 shows porcine satellite cells were treated with 1 of 3 treatments with or without 50 nM rapamycin. The CON treatment consisted of low-glucose Dulbecco's Modified Eagle Medium supplemented with 2% (vol/vol) fetal bovine serum; IGF-1, CON media plus 5 ng/mL active IGF-1; COMBO, CON media plus 5 ng/mL active IGF-1, 3 mM L-glutamine, 1 mM L-leucine, and 1 mM L-arginine.^{a,b,c} Means with different superscripts differ P<0.0001.

FIG. 9 shows avian satellite cells treated with 1 of 6 treatments. The CON treatment consisted of low-glucose Dulbecco's Modified Eagle Medium supplemented with 2% (vol/vol) fetal bovine serum; IGF-1, CON media plus 5 ng/g active IGF-1; COMBO, CON media plus 5 ng/mL active IGF-1, 3 mM L-glutamine, 1 mM L-leucine, and 1 mM L-arginine; GLUT, CON media plus 3 mM L-glutamine; ARG, CON media plus 1 mM L-arginine; LEU, CON media plus 1 mM L-leucine.^{a,b,c} Means with different superscripts differ P<0.05.

FIG. 10 shows body weights at 42 days. Phase 1 diets were fed from day 0-7, phase 2 diets were fed from day 8-21, and phase 3 diets were fed from day 22-42 of the trial. The control diet did not contain any additives to the base diet (CON), a positive control that contained betaGRO® at 2.5 kg per ton in phase 1, and 1.5 kg per ton in phase 2 (BG), a treatment that contained the supplement at 2.5 kg per ton in phase 1 and 1.5 kg per ton in phase 2 (CT1), a treatment that contained the supplement at 350 g per ton in all three phases (CT2), and a treatment that contained the supplement at 175 g per ton in all three phases (CT3).

FIG. 11 shows feed conversion ratio at 42 days. Phase 1 diets were fed from day 0-7, phase 2 diets were fed from day 8-21, and phase 3 diets were fed from day 22-42 of the trial. The control diet did not contain any additives to the base diet (CON), a positive control that contained betaGRO® at 2.5 kg per ton in phase 1, and 1.5 kg per ton in phase 2 (BG), a treatment that contained the supplement at 2.5 kg per ton in phase 1 and 1.5 kg per ton in phase 2 (CT1), a treatment that contained the supplement at 350 g per ton in all three phases (CT2), and a treatment that contained the supplement at 175 g per ton in all three phases (CT3).

FIG. 12 shows fecal diarrhea scores. Phase 1 diets were fed from day 0-7, phase 2 diets were fed from day 8-21, and phase 3 diets were fed from day 22-42 of the trial. The control diet did not contain any additives to the base diet (CON), a positive control that contained betaGRO® at 2.5 kg per ton in phase 1, and 1.5 kg per ton in phase 2 (BG), a treatment that contained the supplement at 2.5 kg per ton in phase 1 and 1.5 kg per ton in phase 2 (CT1), a treatment that contained the supplement at 350 g per ton in all three phases (CT2), and a treatment that contained the supplement at 175 g per ton in all three phases (CT3).

FIG. 13 shows body weights at 84 days. The starter diets were fed from day 0-28, grower diets were fed days 29-56, and finisher diets were fed days 57-84 of the trial. The control diet did not contain any additives to the base diet (CON), a treatment that contained the supplement at 300 g per ton in the starter, and 50 g per ton in phase 2 (CT1), a treatment that contained the supplement at 600 g per ton in the starter, and 100 g per ton in phase 2 (CT2), a treatment that contained the supplement at 600 g per ton in the starter, and 50 g per ton in phase 2 (CT3).

FIG. 14 shows feed conversion at 84 days. The starter diets were fed from day 0-28, grower diets were fed days 29-56, and finisher diets were fed days 57-84 of the trial. The control diet did not contain any additives to the base diet (CON), a treatment that contained the supplement at 300 g per ton in the starter, and 50 g per ton in phase 2 (CT1), a treatment that contained the supplement at 600 g per ton in the starter, and 100 g per ton in phase 2 (CT2), a treatment that contained the supplement at 600 g per ton in the starter, and 50 g per ton in phase 2 (CT3).

FIG. 15 shows lesions scores and E coli prevalence at 84 days. The starter diets were fed from day 0-28, grower diets were fed days 29-56, and finisher diets were fed days 57-84 of the trial. The control diet did not contain any additives to the base diet (CON), a treatment that contained the supplement at 300 g per ton in the starter, and 50 g per ton in phase 2 (CT1), a treatment that contained the supplement at 600 g per ton in the starter, and 100 g per ton in phase 2 (CT2), a treatment that contained the supplement at 600 g per ton in the starter, and 50 g per ton in phase 2 (CT3).

FIG. 16 shows Salmonella incidence and Clostridium perfringes prevalence. The starter diets were fed from day 0-28, grower diets were fed days 29-56, and finisher diets were fed days 57-84 of the trial. The control diet did not contain any additives to the base diet (CON), a treatment that contained the supplement at 300 g per ton in the starter, and 50 g per ton in phase 2 (CT1), a treatment that contained the supplement at 600 g per ton in the starter, and 100 g per ton in phase 2 (CT2), a treatment that contained the supplement at 600 g per ton in the starter, and 50 g per ton in phase 2 (CT3).

FIG. 17 shows illeal villi height at day 84. The starter diets were fed from day 0-28, grower diets were fed days 29-56, and finisher diets were fed days 57-84 of the trial. The control diet did not contain any additives to the base diet (CON), a treatment that contained the supplement at 300 g per ton in the starter, and 50 g per ton in phase 2 (CT1), a treatment that contained the supplement at 600 g per ton in the starter, and 100 g per ton in phase 2 (CT2), a treatment that contained the supplement at 600 g per ton in the starter, and 50 g per ton in phase 2 (CT3).

FIG. 18 shows feed conversion at 3 weeks. Control with no additives added to a complete basal diet (CON), and three treatments that had the supplement added to the basal diet at a of 0.000050, 0.00015, and 0.00045 g/ton of feed (CT 0.05, CT 0.15, and CT 0.45, respectively).

FIG. 19 shows hematocrit level and serum protein level at 3 weeks. Control with no additives added to a complete basal diet (CON), and three treatments that had the supplement added to the basal diet at a of 0.000050, 0.00015, and 0.00045 g/ton of feed (CT 0.05, CT 0.15, and CT 0.45, respectively).

FIG. 20 shows lysozyme and cortisol concentrations at 3 weeks. Control with no additives added to a complete basal diet (CON), and three treatments that had the supplement added to the basal diet at a of 0.000050, 0.00015, and 0.00045 g/ton of feed (CT 0.05, CT 0.15, and CT 0.45, respectively).

FIG. 21 shows sow body weith condition score at farrowing. The control diet did not contain any additives to the base diet (CON), a diet that contained the supplement at 1000 ppm or 1-kg per METRIC TON of feed (CT1), a diet that contained the supplement at 350 ppm or 0.35-kg per METRIC TON of feed (CT2), and a diet that contained the supplement at 100 ppm or 0.10-kg per METRIC TON of feed (CT3). There was a 7-10-day acclimation period prior to sow estrus, and trial day 0 was the time of estrus.

FIG. 22 shows the weaning to estrus interval in days. The control diet did not contain any additives to the base diet (CON), a diet that contained the supplement at 1000 ppm or 1-kg per METRIC TON of feed (CT1), a diet that contained the supplement at 350 ppm or 0.35-kg per METRIC TON of feed (CT2), and a diet that contained the supplement at 100 ppm or 0.10-kg per METRIC TON of feed (CT3). There was a 7-10-day acclimation period prior to sow estrus, and trial day 0 was the time of estrus.

FIG. 23 shows the piglet average daily weight gain. The control diet did not contain any additives to the base diet (CON), a diet that contained the supplement at 1000 ppm or 1-kg per METRIC TON of feed (CT1), a diet that contained the supplement at 350 ppm or 0.35-kg per METRIC TON of feed (CT2), and a diet that contained the supplement at 100 ppm or 0.10-kg per METRIC TON of feed (CT3). There was a 7-10-day acclimation period prior to sow estrus, and trial day 0 was the time of estrus. The piglets were fed a common diet to match the piglet's nutritional needs in the nursery and finishing phases.

FIG. 24 shows the body weight gain of piglets at 126 days. The control diet did not contain any additives to the base diet (CON), a diet that contained the supplement at 1000 ppm or 1-kg per METRIC TON of feed (CT1), a diet that contained the supplement at 350 ppm or 0.35-kg per METRIC TON of feed (CT2), and a diet that contained the supplement at 100 ppm or 0.10-kg per METRIC TON of feed (CT3). There was a 7-10-day acclimation period prior to sow estrus, and trial day 0 was the time of estrus. The piglets were fed a common diet to match the piglet's nutritional needs in the nursery and finishing phases.

FIG. 25 shows the feed conversion ratio of piglets at 126 days. The control diet did not contain any additives to the base diet (CON), a diet that contained the supplement at 1000 ppm or 1-kg per METRIC TON of feed (CT1), a diet that contained the supplement at 350 ppm or 0.35-kg per METRIC TON of feed (CT2), and a diet that contained the supplement at 100 ppm or 0.10-kg per METRIC TON of feed (CT3). There was a 7-10-day acclimation period prior to sow estrus, and trial day 0 was the time of estrus. The piglets were fed a common diet to match the piglet's nutritional needs in the nursery and finishing phases.

### DETAILED DESCRIPTION

The present disclosure provides a supplement that includes the amino acids L-glutamine (also referred to herein as Gln or Q), L-leucine (also referred to herein as Leu or L) and L-arginine (also referred to herein as Arg or R). The amino acids can be present in the supplement as a free amino acid, e.g., not covalently attached to other amino acids to form a protein, as a salt of the amino acid, or a mixture of both free and salt forms of the amino acids. Thus, the glutamine in a supplement can be entirely the free form, entirely a salt form, or a mixture of both; the leucine in the supplement can be entirely the free form, entirely a salt form, or a mixture of both; and the arginine in the supplement can be entirely the free form, entirely a salt form, or a mixture of both. Unless the context indicates otherwise, reference herein to an amino acid includes the free amino acid and a salt of the amino acid. The amino acids glutamine, leucine, and arginine and salt forms thereof are readily available.

In one embodiment, the three amino acids are present in a supplement of the present disclosure in a known ratio. In one embodiment, the ratio of the three amino acids is based on the ratio of moles (molar ratio) of each amino acid.

The molar ratio of glutamine to leucine and arginine can vary from 2 to 4, and includes 2, 2.1, 2.2, 2.3, 2.4, 2.5, 2.6, 2.7, 2.8, 2.9, 3.0 3.1, 3.2, 3.3, 3.4, 3.5, 3.6, 3.7, 3.8, 3.9, and 4. The molar ratio of leucine to glutamine and arginine can vary from 0.5 to 2, and includes 0.5, 0.6, 0.7, 0.8, 0.9, 1, 1.1, 1.2, 1.3, 1.4, 1.5, 1.6, 1.7, 1.8, 1.9, and 2. The molar ratio of arginine to glutamine and leucine can vary from 0.5 to 3, and includes 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, and 3. Non-limiting examples of molar ratios of Q:L:R that can be present in a supplement of the present disclosure include 2:1:1, 4:1:1, 3:0.5:1, 3:1.5:1, 3:1:0.5, and 3:1:1.5. In one embodiment, the ratio of Q:L:R is 3:1:1.

Typically, to determine the amount of each amino acid needed to achieve a desired ratio the number of moles of each amino acid can be converted to mass, and those amounts of the amino acids combined. For instance, when the ratio of Q:L:R is 3:1:1, 44 grams of L-glutamine, 13 grams L-leucine, and 17 grams of L-arginine can be combined to yield the correct ratio of amino acids.

The supplement also includes active insulin-like growth factor 1 (IGF-1, also referred to herein as IGF). Whether a protein is an IGF-1 can be easily determined by the skilled person. For instance, polyclonal and monoclonal antibodies that specifically bind to IGF-1 are commercially available and specifically react with IGF-1 from various species including human, equine, canine, bovine, porcine, ovine, and avian. These readily available antibodies lack cross-reactivity and/or interference by other closely related proteins and binding proteins. A single antibody or a panel of antibodies that recognizes different regions of an IGF, such as N-terminal, C-terminal, or amino acids present between the ends of the protein, may be used to determine whether a protein is an IGF-1 protein. Methods for determining whether an IGF-1 protein is active are known in the art and routine.

IGFs are proteins with high sequence similarity to insulin, but unlike insulin, IGFs associate with distinct binding proteins present in serum and other biological fluids (Baxter, 2000, Am J Physiol Endocrinol Metab, 278: E967-E976; Hwa et al., 1999, Endocrine Reviews, 20(6):761-787). Most IGF present in products derived from an animal, such as, but not limited to, blood and blood-derived products, milk and milk-derived products, and colostrum and colostrum-derived products, is bound to a binding protein. However, since these binding proteins inhibit the activity of IGF, most IGF present in animal derived products is inactive due to its being bound to a binding protein. For instance, less than 1% of IGF-1 in plasma is not bound to a binding protein (Carel et al., Safety of Recombinant Human Growth Hormone, In: Current Indications for Growth Hormone Therapy, 2nd rev. ed., vol. ed.: Hindmarsh, Karger, Switzerland, p. 48).

IGF is considered to be active if it is not bound to a binding protein and is considered to be inactive if it is bound to a binding protein. Active IGF is often referred in the art as free, unbound, bioactive, and/or active. Methods for measuring the concentration of active IGF are known to the skilled person and are routine. Assays, including solid phase sandwich ELISA assays, are commercially available that permit measurement of IGF that is not bound to a binding protein (e.g., R&D Systems, Minneapolis, Minn., catalog number DG100).

IGF useful in the supplements and methods of the present disclosure is obtainable from various sources. In one embodiment, a source is a natural source, such as a biological material from an animal. Examples of animals include, but are not limited to, vertebrates, including but not limited to bovine, porcine, avian, equine, and ovine animals. The amino acid sequence of IGF from different animals is highly conserved and as a result IGF from one type of animal is typically biologically active in other animals. For instance, the examples show that porcine IGF functions in other animals including chickens, turkeys, and fish such as sea bass.

Examples of biological materials that can be used as a source of IGF include, but are not limited to, blood and blood-derived products (e.g., whole blood, red blood cells, plasma, and derivatives thereof); milk and milk products (e.g., liquid milk, powdered milk, cheese, whey and whey products, curd, cheese, casein, lactose, milk fat, and derivatives thereof); colostrum and colostrum-derived products (e.g., liquid colostrum, dried colostrum); egg and egg-derived products (e.g., egg yolk, egg whites, egg membranes), bodily fluids (e.g., saliva, semen), and tissues (e.g., mucosa tissue, intestinal tissue, embryonic tissue). Biological materials useful for producing a supplement with active IGF is readily available commercially.

In one embodiment, IGF useful in the methods described herein is produced using recombinant techniques, or chemically or enzymatically synthesized. Recombinant production can be accomplished in essentially any expression system, including but not limited to prokaryotic, such as bacterial (e.g., E. coli), and eukaryotic (e.g., yeast) systems. Polynucleotide sequences encoding active IGF are readily available and methods for producing recombinant IGF are known and routine. IGF produced using recombinant techniques, or chemically or enzymatically synthesized is typically active because it is not exposed to a binding protein.

In one embodiment, the amount of the amino acid glutamine in the supplement can be at least 0.5 grams glutamine/kilogram (g/kg) of the supplement, at least 5 g/kg, at least 10 g/kg, at least 20 g/kg, at least 30 g/kg, at least 40 g/kg, at least 50 g/kg, or at least 60 g/kg of the supplement. In one embodiment, the amount of the amino acid glutamine in the supplement can be no greater than 500 g/kg of the supplement, no greater than 400 g/kg, no greater than 300 g/kg, no greater than 200 g/kg, no greater than 100 g/kg, no greater than 80 g/kg, no greater than 60 g/kg, no greater than 50 g/kg, no greater than 40 g/kg, or no greater than 30 g/kg of the supplement. For instance, the amount of glutamine in a supplement of the present disclosure can range from 0.5 g/kg to 500 g/kg, 10 g/kg to 100 g/kg, 20 g/kg to 80 g/kg, or 30 g/kg to 60 g/kg.

In one embodiment, the amount of the amino acid leucine in the supplement can be at least 0.1 g leucine/kg of the supplement, at least 1 g/kg, at least 5 g/kg, at least 10 g/kg, at least 15 g/kg, at least 20 g/kg, or at least 25 g/kg of the supplement. In one embodiment, the amount of the amino acid leucine in the supplement can be no greater than 100 g/kg of the supplement, no greater than 75 g/kg, no greater than 50 g/kg, no greater than 40 g/kg, no greater than 30 g/kg, no greater than 25 g/kg, no greater than 20 g/kg, no greater than 15 g/kg, or no greater than 10 g/kg of the supplement. For instance, the amount of leucine in a supplement of the present disclosure can range from 0.1 g/kg to 100 g/kg, 1 g/kg to 75 g/kg, or 5 g/kg to 50 g/kg.

In one embodiment, the amount of the amino acid arginine in the supplement can be at least 0.1 g arginine/kg of the supplement, at least 1 g/kg, at least 10 g/kg, at least 20 g/kg, at least 30 g/kg, at least 40 g/kg, or at least 50 g/kg of the supplement. In one embodiment, the amount of the amino acid arginine in the supplement can be no greater than 200 g/kg of the supplement, no greater than 100 g/kg, no greater than 75 g/kg, no greater than 50 g/kg, no greater than 40 g/kg, no greater than 30 g/kg, no greater than 20 g/kg, no greater than 10 g/kg, or no greater than 1 g/kg of the supplement. For instance, the amount of arginine in a supplement of the present disclosure can range from 0.1 g/kg to 200 g/kg, or 1 g/kg to 100 g/kg.

In one embodiment, the amount of active IGF-1 in the supplement can be at least 100 nanograms/kilogram (ng/kg) of the supplement, at least 200 μg/kg, at least 300 μg/kg, at least 400 μg/kg, at least 500 μg/kg, at least 600 μg/kg, or at least 700 μg/kg of the supplement. In one embodiment, the amount of active IGF-1 in the supplement can be no greater than 2000 micrograms/kilogram (μg/kg) of the supplement, no greater than 1750 μg/kg, no greater than 1500 μg/kg, no greater than 1250 μg/kg, no greater than 1000 μg/kg, no greater than 800 μg/kg, no greater than 700 μg/kg, no greater than 600 μg/kg, no greater than 500 μg/kg, no greater than 400 μg/kg, no greater than 300 μg/kg, or no greater than 200 μg/kg of the supplement. In one embodiment, the amount of IGF-1 in a supplement of the present disclosure can range from 100 μg/kg to 2000 μg/kg, 200 μg/kg to 1750 μg/kg, 300 μg/kg to 1500 μg/kg, or 400 μg/kg to 1250 μg/kg.

A supplement of the present disclosure can also include other components including, but not limited to, grains, proteins, vitamins, minerals, preservatives, antibiotics, pigments, stabilizers including heat stabilizers, and other additives. In one embodiment, the supplement includes yeast. In one embodiment, a component includes an extract of the cell in which the IGF was expressed. For instance, if recombinant IGF is expressed in yeast the yeast containing the IGF can be lysed and the resulting extract used. In one embodiment, a component can be inactive.

In one embodiment, the supplement includes glutamine at 30 g/kg to 60 g/kg (e.g., 44 g/kg), leucine at 5 g/kg to 50 g/kg (e.g., 13 g/kg), arginine at 1 g/kg to 100 g/kg (e.g., 17 g/kg), active IGF at 300 μg/kg to 1500 μg/kg (e.g., 450 μg/kg), and yeast (e.g., 93 wt %).

In one embodiment, the supplement includes active IGF that is produced using recombinant system, such as a prokaryotic expression system (e.g., E. coli) or eukaryotic system (e.g., yeast). The IGF can be from any source, for example equine, canine, bovine, porcine, ovine, or avian. In one embodiment, a recombinant IGF is porcine.

In one embodiment, a supplement of the present disclosure is used as a supplement for addition to a food product or water for an animal. Accordingly, a supplement of the present disclosure can take any form that is useful for adding to a food product or to water. For instance, the supplement can be in the form of a liquid, emulsion, powder (e.g., spray dried powder), cake, meal, pellets, crumbles, granules, and the like.

Also provided by the present disclosure is a composition that includes the supplement. In one embodiment, the composition is a food product. As used herein, a “food product” is a compound or mixture of compounds that can be consumed by an animal and that provides nutrition for that animal. In one embodiment, a food product is a feed for animal use, for instance, for feeding domesticated animals such as companion animals including, but not limited to, canine and feline animals, and livestock including, but not limited to, bovine, porcine, avian, equine, and ovine animals. In another embodiment, the animal is an aquatic animal used in aquaculture, such as but not limited to fish including sea bass, shrimp, or eel. A food product may be solid, semi-solid, or liquid. Various food products useful for administering to animals are available. In one embodiment, a food product is one designed to feed an animal that is used for meat production. In one embodiment, a food product is one designed to feed an animal that is used for egg production. In one embodiment, the supplement is added to an animal's water source.

The amount of a supplement of the present disclosure that is added to a composition, such as a food product, is sufficient to be useful in the methods described herein.

In one embodiment, the amount of the supplement added to a food product, such as an animal feed or to water, is at least 0.0000005 grams/ton (g/ton) of the food product, at least 0.000005 g/ton, at least 0.00005 g/ton, at least 0.0005 g/ton, or at least 0.005 g/ton. In one embodiment, the amount of the supplement added to a food product is no greater than 0.05 g/ton of the supplement, no greater than 0.005 g/ton, no greater than 0.0005 g/ton, no greater than 0.00005 g/ton, or no greater than 0.000005 g/ton. In one embodiment, the amount of the supplement of the present disclosure in a food product can range from 0.000005 g/ton to 0.005 g/ton, 0.00005 g/ton to 0.005 g/ton or 0.00005 g/ton to 0.0005 g/ton.

In one embodiment, the amount of the supplement added to a food product is at least 5 grams/ton (g/ton) of the food product, at least 25 g/ton, at least 50 g/ton, at least 100 g/ton, at least 250 g/ton, at least 500 g/ton, or at least 750 g/ton. In one embodiment, the amount of the supplement added to a food product is no greater than 2750 g/ton of the supplement, no greater than 2600 g/ton, no greater than 2400 g/ton, no greater than 2250 g/ton, no greater than 2000 g/ton, no greater than 1750 g/ton, no greater than 1500 g/ton, no greater than 1250 g/ton, no greater than 1000 g/ton, no greater than 750 g/ton, no greater than 500 g/ton, no greater than 250 g/ton, no greater than 100 g/ton, or no greater than 50 g/ton. In one embodiment, the amount of the supplement of the present disclosure in a food product can range from 5 g/ton to 2750 g/ton, or 50 g/ton to 2600 g/ton.

In one embodiment, the amount of the amino acid glutamine in the food product can be at least 0.0000002 wt % of the food product, at least 0.000002 wt %, at least 0.00002 wt %, or at least 0.0002 wt % of the food product. In one embodiment, the amount of the amino acid glutamine in the food product can be no greater than 0.1 wt % of the supplement, no greater than 0.01 wt %, or no greater than 0.001 wt % of the food product. In one embodiment, the amount of glutamine in a food product of the present disclosure can range from 0.0000002 wt % to 0.1 wt %, from 0.000002 wt % to 0.1 wt %, from 0.00002 wt % to 0.1 wt %, from 0.0002 wt % to 0.1 wt %, from 0.002 wt % to 0.1 wt %, or from 0.02 wt % to 0.1 wt %.

In one embodiment, the amount of the amino acid leucine in the food product can be at least 0.00000006 wt % of the food product, at least 0.0000006 wt %, at least 0.000006 wt %, at least 0.00006 wt %, or at least 0.0006 wt % of the food product. In one embodiment, the amount of the amino acid leucine in the food product can be no greater than 0.03 wt % of the supplement, no greater than 0.003 wt %, or no greater than 0.0003 wt % of the food product. In one embodiment, the amount of leucine in a food product of the present disclosure can range from 0.00000006 wt % to 0.03 wt %, from 0.0000006 wt % to 0.03 wt %, from 0.000006 wt % to 0.03 wt %, from 0.00006 wt % to 0.03 wt %, from 0.0006 wt % to 0.03 wt %, or from 0.006 wt % to 0.03 wt %.

In one embodiment, the amount of the amino acid arginine in the food product can be at least 0.00000006 wt % of the food product, at least 0.0000006 wt %, at least 0.000006 wt %, at least 0.00006 wt %, or at least 0.0006 wt % of the food product. In one embodiment, the amount of the amino acid arginine in the food product can be no greater than 0.06 wt % of the supplement, no greater than 0.006 wt %, no greater than 0.0006 wt % of the food product. In one embodiment, the amount of arginine in a food product of the present disclosure can range from 0.00000006 wt % to 0.06 wt %, from 0.0000006 wt % to 0.06 wt %, from 0.000006 wt % to 0.06 wt %, from 0.00006 wt % to 0.06 wt %, from 0.0006 wt % to 0.06 wt %, or from 0.006 wt % to 0.06 wt %.

In one embodiment, the total weight percent (wt %) of the L-glutamine or salt thereof, the L-leucine or salt thereof, and the L-arginine or salt thereof in the composition added by the supplement is less than the total wt % of any other free natural amino acid or salt thereof in the composition. In one embodiment, the total weight percent (wt %) of the L-glutamine or salt thereof, the L-leucine or salt thereof, and the L-arginine or salt thereof in the composition added by the supplement is less than the total wt % of the combination of other free natural amino acids or salts thereof in the composition.

The present disclosure also provides methods for using a supplement described herein. In one embodiment, the method includes making a food product described herein. The method includes combining the supplement with a food product at an amount as described herein.

In one embodiment, the method includes administering the food product to an animal.

Administering includes making the food product available to the animal. The administering can begin as soon after birth as possible. For instance, the food product can be made available immediately after birth of a non-mammalian animal, e.g., poultry. In the case of a mammalian animal the food product can be made available when the animal is weaned. The food product can be administered to the animal throughout its life or at different stages, such as before weaning, after weaning and before adulthood, and/or adulthood. In some embodiments, the food product can be administered as one or more rations. Rations for livestock can be divided into diets that are provided to an animal at different times during its lifetime and are often referred to in the art as a prestarter ration, a starter ration, a grower ration, and a finisher ration. In one embodiment, a food is available ad libitum.

In one embodiment, when the animal is porcine, such as a piglet, sow, gilt, and/or boar, the supplement is present in the composition at a range of from 10 grams supplement/ton of food to 2.75 kg/ton, or 100 g/ton of food to 2.5 kg/ton. In one embodiment, when the animal is a turkey, the supplement is present in the composition at a range of from 50 grams supplement/ton of food to 1200 g/ton, or 100 g/ton to 600 g/ton. In one embodiment, when the animal is a layer chicken, the supplement is present in the composition at a range of from 30 grams supplement/ton of food to 90 g/ton or, 50 g/ton to 70 g/ton. In one embodiment, when the animal is a chicken broiler, the supplement is present in the composition at a range of from 25 grams supplement/ton of food to 600 g/ton, or 50 g/ton to 300 g/ton. In one embodiment, when the animal is a fish such as Asian sea bass, the supplement is present in the composition at a range of from 0.000050 grams supplement/ton of food to 0.0005 g/ton, or 0.000050 g/ton to 0.00045 g/ton.

In one embodiment, the method is for improving the performance of an animal, improving a processing factor of an animal, increasing an intestinal health factor of an animal, or a combination thereof. In one embodiment, the animal is livestock that is housed in a high stress environment. As used herein, a “high stress environment” refers to an environment that is not optimal for growth of the animal. Examples of environmental conditions that are high stress include, but are not limited to, an undesirable ambient temperature (either too high or too low), and the presence of a pathogen in the environment. As used herein, a “low stress environment” refers to an environment that is optimal for growth of the animal. Environmental conditions optimal for the growth of an animal are known to the person of ordinary skill.

Examples of performance include but are not limited to increased body weight, increased feed conversion, reduced mortality rate, and increased flock uniformity as measured by, for instance, weight variation. Typically, determining whether there is a change in body weight, feed conversion, mortality rate, flock uniformity, immune response, gut health, and/or ability to respond to systemic stress is done by comparison of a population of animals receiving the supplement with a population of animals not receiving the supplement. In one embodiment, a method of the present disclosure results in an increase of body weight of animals compared to animals in a similar environment but not fed the food product containing the supplement. In one embodiment, a method of the present disclosure results in an increase in feed conversion in animals compared to animals in a similar environment but not fed the food product containing the supplement. In one embodiment, a method of the present disclosure results in a decrease in mortality in animals compared to animals in a similar environment but not fed the food product containing the supplement. In one embodiment, a method of the present disclosure results in increased flock uniformity in animals compared to animals in a similar environment but not fed the food product containing the supplement.

In one embodiment, the increase of body weight, increase of feed conversion, increase of flock uniformity, decrease of mortality rate, immune response, gut health, and/or ability to respond to systemic stress of animals is a statistically significant change compared to animals in a similar environment but not fed the food product containing the supplement. In one embodiment, the increase of body weight, increase of feed conversion, increase of flock uniformity, decrease of mortality rate, immune response, gut health, and/or ability to respond to systemic stress is a change of at least 0.1%, at least 0.25%, at least 0.5%, at least 1%, at least 3%, at least 5%, at least 7%, at least 9%, at least 11%, or at least 13% compared to animals in a similar environment but not fed the food product containing the supplement.

Examples of processing factors include but are not limited to chilled carcass yield and total breast meat yield as a percentage of live weight. Typically, determining whether there is a change in chilled carcass yield and/or an increased total breast meat yield is done by comparison of a population of animals receiving the supplement with a population of animals not receiving the supplement. In one embodiment, a method of the present disclosure results in an increase of chilled carcass yield of animals compared to animals in a similar environment but not fed the food product containing the supplement. In one embodiment, a method of the present disclosure results in an increase of total breast meat yield as a percentage of live weight in animals compared to animals in a similar environment but not fed the food product containing the supplement.

In one embodiment, the increase of chilled carcass yield and/or increase of total breast meat yield as a percentage of live weight is a statistically significant change compared to animals in a similar environment but not fed the food product containing the supplement. In one embodiment, the increase of chilled carcass yield and/or increase of total breast meat yield as a percentage of live weight of animals is a change of at least 0.1%, at least 0.25%, at least 0.5%, at least 1%, at least 3%, at least 5%, at least 7%, at least 9%, at least 11%, or at least 13% compared to animals in a similar environment but not fed the food product containing the supplement.

Examples of intestinal health factors include but are not limited to villi development, intestinal lesions, intestinal bacteria, and diarrhea. Typically, determining whether there is a change in villi development, intestinal lesions, intestinal bacterial, and/or diarrhea is done by comparison of a population of animals receiving the supplement with a population of animals not receiving the supplement. In one embodiment, a method of the present disclosure results in an increase of villi cell height and/or increase crypt depth of animals compared to animals in a similar environment but not fed the food product containing the supplement. In one embodiment, a method of the present disclosure results in a decrease of intestinal lesions in animals compared to animals in a similar environment but not fed the food product containing the supplement. In one embodiment, a method of the present disclosure results in a decrease of intestinal bacteria (e.g., E. coli, Salmonella, and/or Clostridia) in animals compared to animals in a similar environment but not fed the food product containing the supplement. In one embodiment, a method of the present disclosure results in a decrease of diarrhea in animals compared to animals in a similar environment but not fed the food product containing the supplement.

In one embodiment, the change in villi development, decrease of intestinal lesions, and/or decrease of intestinal bacteria is a statistically significant change compared to animals in a similar environment but not fed the food product containing the supplement. In one embodiment, the change in villi development, decrease of intestinal lesions, and/or decrease of intestinal bacteria is a change of at least 0.1%, at least 0.25%, at least 0.5%, at least 1%, at least 3%, at least 5%, at least 7%, at least 9%, at least 11%, or at least 13% compared to animals in a similar environment but not fed the food product containing the supplement.

In one embodiment, the food product containing the supplement is provided to an animal, such as a sow, after offspring are weaned and until estrus. The food product can result in in reducing the interval between weaning and estrus. The reduction in the weaning to estrus interval can be a change of at least 0.1%, at least 0.25%, at least 0.5%, at least 1%, at least 3%, at least 5%, at least 7%, at least 9%, at least 11%, or at least 13% compared to animals in a similar environment but not fed the food product containing the supplement, or by at least 0.5, at least 1, at least 1.5, or at least 2 days compared to animals in a similar environment but not fed the food product containing the supplement.

In one embodiment, the food product containing the supplement is provided to an animal, such as a piglet. Addition of the supplement to the diet can result in increased average daily gain, increased 126-day body weight, reduced fecal diarrhea, and/or improved feed conversion. The increased average daily gain, increased body weight such as body weight at 126 days after weaning, reduced fecal diarrhea, and/or improved feed conversion can be a change of at least 0.1%, at least 0.25%, at least 0.5%, at least 1%, at least 3%, at least 5%, at least 7%, at least 9%, at least 11%, or at least 13% compared to animals in a similar environment but not fed the food product containing the supplement.

In one embodiment, the food product containing the supplement is provided to an animal, such as an avian layer, for instance, a chicken layer. Addition of the supplement to the diet can result in increased body weight, improved health as measured by reduced intestinal lesions and/or increased fat pad as a percentage of body weight, increased egg production, increased total eggs laid, and/or improved feed conversion. Surprisingly, the increased egg production and increased total eggs laid did not result in reduced egg weights and reduced eggshell weights. The increased body weight, improved health as measured by reduced intestinal lesions and/or increased fat pad as a percentage of body weight, increased egg production, increased total eggs laid, and/or improved feed conversion can be a change of at least 0.1%, at least 0.25%, at least 0.5%, at least 1%, at least 3%, at least 5%, at least 7%, at least 9%, at least 11%, or at least 13% compared to animals in a similar environment but not fed the food product containing the supplement.

In one embodiment, the food product containing the supplement is provided to an animal, such as a fish, such as Asian sea bass. Addition of the supplement to the diet can result in an increased immune response as indicated by a greater hematocrit level, a greater concentration of serum protein, improved gut health as indicated by greater lysozyme concentrations, and/or greater ability to respond to system stress as indicated by increased serum cortisol concentrations. The increased immune response, greater concentration of serum protein, improved gut health, and/or greater ability to respond to system stress can be a change of at least 0.1%, at least 0.25%, at least 0.5%, at least 1%, at least 3%, at least 5%, at least 7%, at least 9%, at least 11%, or at least 13% compared to animals in a similar environment but not fed the food product containing the supplement.

### EXEMPLARY EMBODIMENTS

Embodiment 1. A supplement comprising free amino acids L-glutamine or a salt thereof, L-leucine or a salt thereof, and L-arginine or a salt thereof, and active IGF-1, wherein the L-glutamine or salt thereof, L-leucine or salt thereof, and L-arginine or salt thereof are present at a ratio of 2-4:1:1, 3:0.5-2:1, or 3:1:0.5-3.

Embodiment 2. The supplement of embodiment 1 wherein the at least one of the L-glutamine, the L-leucine, and the L-arginine is in a salt form.

Embodiment 3. The supplement of any one of embodiments 1-2 wherein the amount of L-glutamine or a salt thereof is at least 0.5 grams to no greater than 500 grams amino acid/kilogram supplement.

Embodiment 4. The supplement of any one of embodiments 1-3 wherein the amount of L-leucine or a salt thereof is at least 0.1 grams to no greater than 100 grams amino acid/kilogram supplement.

Embodiment 5. The supplement of any one of embodiments 1˜4 wherein the amount of L-arginine or a salt thereof is at least 0.1 grams to no greater than 200 grams amino acid/kilogram supplement.

Embodiment 6. The supplement of any one of embodiments 1-5 wherein the amount of active IGF-1 is at least 100 micrograms to no greater than 2000 micrograms active IGF-1/kilogram supplement.

Embodiment 7. The supplement of any one of embodiments 1-6 wherein the amount of L-glutamine or a salt thereof is at least 0.5 grams to no greater than 500 grams amino acid/kilogram supplement, the amount of L-leucine or a salt thereof is at least 0.1 grams to no greater than 100 grams amino acid/kilogram supplement, the amount of L-arginine or a salt thereof is at least 0.1 grams to no greater than 200 grams amino acid/kilogram supplement, and the amount of active IGF-1 is at least 100 grams to no greater than 2000 microgram IGF-1/kilogram supplement.

Embodiment 8. The supplement of any one of embodiments 1-7 wherein the active IGF-1 is recombinant IGF-1.

Embodiment 9. The supplement of any one of embodiments 1-8 further comprising a food product.

Embodiment 10. A food product comprising the supplement of any one of embodiments 1-9 present in the food product at an amount of at least 0.000005 g/ton and no greater than 0.005 g/ton

Embodiment 11. A food product comprising the supplement of any one of embodiments 1-9 present in the food product at an amount of at least 50 grams/ton (g/ton) of the food product and no greater than 2600 g/ton.

Embodiment 12. The food product of embodiment 10 or 11 wherein the total weight percent (wt %) of the L-glutamine or salt thereof, the L-leucine or salt thereof, and the L-arginine or salt thereof is less than the total wt % of other amino acids in the supplement.

Embodiment 13. The food product of any one of embodiments 10-12 wherein one, two, or all three of the L-glutamine, the L-leucine, and the L-arginine is in a salt form.

Embodiment 14. The food product of any one of embodiments 10-13 wherein the amount of L-glutamine or a salt thereof is at least 0.0000002 wt %.

Embodiment 15. The food product of any one of embodiments 10-14 wherein the amount of L-leucine or a salt thereof is at least 0.00000006 wt %.

Embodiment 16. The food product of any one of embodiments 10-15 wherein the amount of L-arginine or a salt thereof is at least 0.00000006 wt %.

Embodiment 17. A method comprising administering to an animal an effective amount of (i) a food product comprising the supplement of any one of embodiments 1-9, or (ii) the food product of any one of embodiments 10-16.

Embodiment 18. A method of improving the performance of an animal comprising administering to the animal (i) a food product comprising the supplement of any one of embodiments 1-9, or (ii) the food product of any one of embodiments 10-16, wherein the improving the performance comprises an increased body weight, an improved feed conversion, a decreased mortality rate, increased flock uniformity, or a combination thereof.

Embodiment 19. A method of improving a processing factor of an animal comprising administering to the animal (i) a food product comprising the supplement of any one of embodiments 1-9, or (ii) the food product of any one of embodiments 10-16, wherein the improving the processing factor is an increased chilled carcass yield, an increased total breast meat yield, or a combination thereof.

Embodiment 20. A method for increasing intestinal health of an animal comprising administering to the animal (i) a food product comprising the supplement of any one of embodiments 1-9, or (ii) the food product of any one of embodiments 10-16, wherein the improving the intestinal health is a decrease in an intestinal bacteria, a decrease in intestinal lesions, a decrease in fecal diarrhea, or a combination thereof.

Embodiment 21. The method of any one of embodiments 17 to 20 wherein the food product comprises the supplement at an amount of at least 50 g/ton.

Embodiment 22. The method of any one of embodiments 17 to 21 wherein the animal is a porcine animal or an avian animal.

Embodiment 23. The method of any one of embodiments 17 to 22 wherein the animal is housed in a high stress environment.

Embodiment 24. A method of reducing time from weaning offspring to return to estrus, comprising administering to the animal (i) a food product comprising the supplement of any one of embodiments 1-9, or (ii) the food product of any one of embodiments 10-16, wherein the time from weaning offspring to return to estrus is reduced compared to animals in a similar environment but not fed the composition.

Embodiment 25. The method of embodiment 24 wherein the animal is a sow.

Embodiment 26. The method of embodiment 24 or 25 wherein the supplement is present at 100 grams supplement/ton of food to 2.5 kg/ton.

Embodiment 27. A method of increasing average daily gain, increasing body weight, reducing fecal diarrhea, and/or improving feed conversion in an animal, comprising administering to the animal (i) a food product comprising the supplement of any one of embodiments 1-9, or (ii) the food product of any one of embodiments 10-16, wherein the increasing average daily gain, increasing body weight, reducing fecal diarrhea, and/or improving feed conversion is a change compared to animals in a similar environment but not fed the food product containing the supplement.

Embodiment 28. The method of embodiment 27 wherein the animal is a piglet.

Embodiment 29. The method of embodiment 27 or 28 wherein the supplement is present at 100 grams supplement/ton of food to 2.5 kg/ton.

Embodiment 30. A method of increasing body weight, improving health as measured by reduced intestinal lesions and/or increased fat pad as a percentage of body weight, increasing egg production, increasing total eggs laid, and/or improving feed conversion in an avian layer, comprising administering to the animal (i) a food product comprising the supplement of any one of embodiments 1-9, or (ii) the food product of any one of embodiments 10-16, wherein the increasing body weight, improving health as measured by reduced intestinal lesions and/or increased fat pad as a percentage of body weight, increasing egg production, increasing total eggs laid, and/or improving feed conversion is a change compared to animals in a similar environment but not fed the food product containing the supplement.

Embodiment 31. The method of embodiment 30 wherein the avian layer is a chicken.

Embodiment 32. The method of embodiment 30 or 31 wherein the supplement is present at 30 grams supplement/ton of food to 90 g/ton.

Embodiment 33. A method of increasing hematocrit level, increasing concentration of serum protein, increasing lysozyme concentrations, and/or increasing serum cortisol concentrations in a fish, comprising administering to the animal (i) a food product comprising the supplement of any one of embodiments 1-9, or (ii) the food product of any one of embodiments 10-16, wherein the increasing hematocrit level, increasing concentration of serum protein, increasing lysozyme concentrations, and/or increasing serum cortisol concentrations is a change compared to animals in a similar environment but not fed the food product containing the supplement.

Embodiment 34. The method of embodiment 33 wherein the fish is Asian sea bass.

Embodiment 35. The method of embodiment 33 or 34 wherein the supplement is present at 0.000050 grams supplement/ton of food to 0.0005 g/ton.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

The Combinatorial Influence of IGF-1, L-Glutamine, L-Leucine, and L-Arginine on Skeletomuscular Driven Growth in Avian and Porcine Species

The usage of bio-active amino acids (AA) and insulin like growth factor-1 (IGF-1) has been researched in depth, as individual AA or particular combinations. Amino acid supplementation typically includes essential amino acids, with lysine and methionine most often being used as supplements for commercial livestock feeds. The current report illustrates the surprising finding that the two non-essential amino L-glutamine and L-arginine, with L-leucine in combination with active IGF-1 is useful in the commercial production of livestock species.

Materials and Methods

Porcine and avian satellite cells were isolated as previously described (Vaughn et al., 2017). Porcine satellite cells (SC) were plated at a density of 5000 cells per cm² and cultured in growth media (GM) consisting of high-glucose Dulbecco's Modified Eagle Medium (Invitrogen, Carlsbad, Calif.) supplemented with 10% (vol/vol) fetal bovine serum (GE Healthcare, Pittsburgh, Pa.), 100 U penicillin/mL, 100 μg of strepomycin/mL, and 20 μg of gentamicin/mL for 3 days to allow SC to reach 70 percent confluence. At this point culture media was replaced with differentiation media (DM) consisting of low-glucose Dulbecco's Modified Eagle Medium (Invitrogen, Carlsbad, Calif.) supplemented with 2% (vol/vol) fetal bovine serum (GE Healthcare, Pittsburgh, Pa.), 100 U penicillan/mL, 100 μg of strepmycin/mL, and 20 μg of gentamicin/mL for 4 days to induce myotube development. At this point differentiated SC were treatments were applied in DM as described below for a 72 hour treatment period.

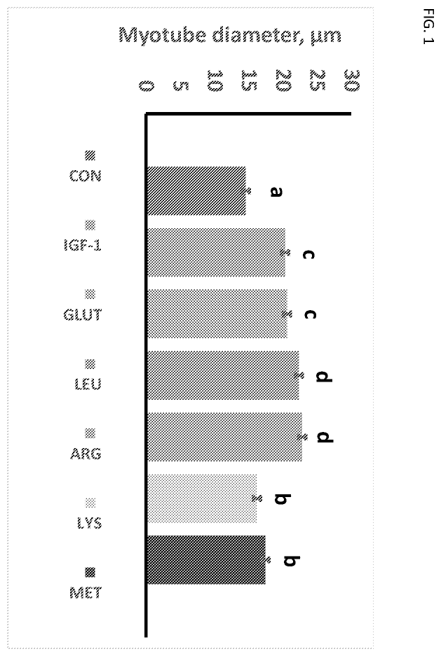

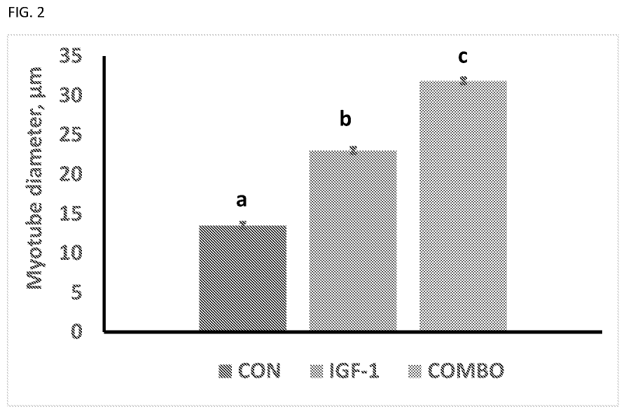

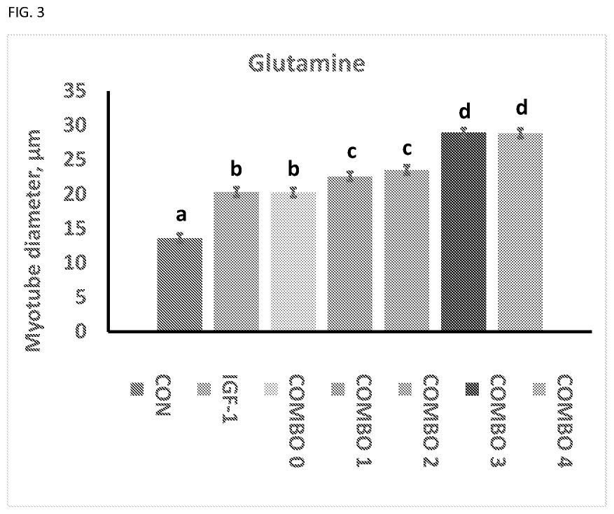

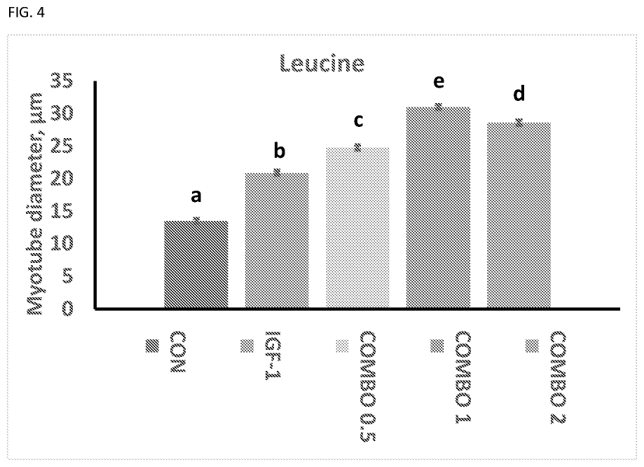

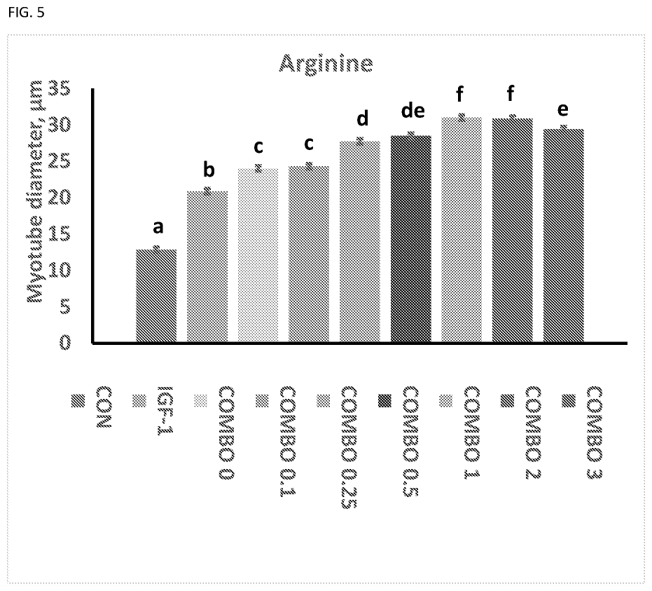

The first series of experiments were conducted on porcine satellite cells with a negative control consisting of DM. Supplementation of a yeast broth 5 ng/mL yeast produced active IGF-1, L-leucine (1 mM), L-arginine (1 mM), L-glutamine (4 mM), L-lysine (10 mM), and L-methionine (10 μM) were evaluated by adding them to DM, and dosage was based on doses reported in the literature, data shown in FIG. 1. Based on these results the subsequent experiment was conducted by combining glutamine, arginine, and leucine at the same dosage as used in the first experiment, data shown in FIG. 2. The next series of experiments were conducted by titrating each individual amino acid to the combination of amino acids, to determine useful concentrations of each amino acid, as outlined in FIGS. 3-7.

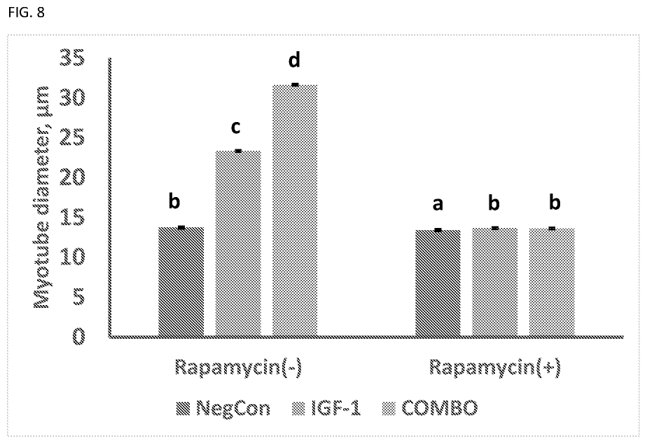

In the previous experiment the concentration and inclusion of amino acids was determined to be 1 mM L-leucine, 1 mM arginine, 3 mM glutamine, added to DM containing 5 ng/mL yeast produced active IGF-1. A pivotal cell signaling pathway that is the potential mode of action here is the mammalian target of rapamycin (mTOR) pathway signaling. To test if this is the mode of action of this combination of amino acids rapamycin was added to the culture medium, data outlined in FIG. 8.

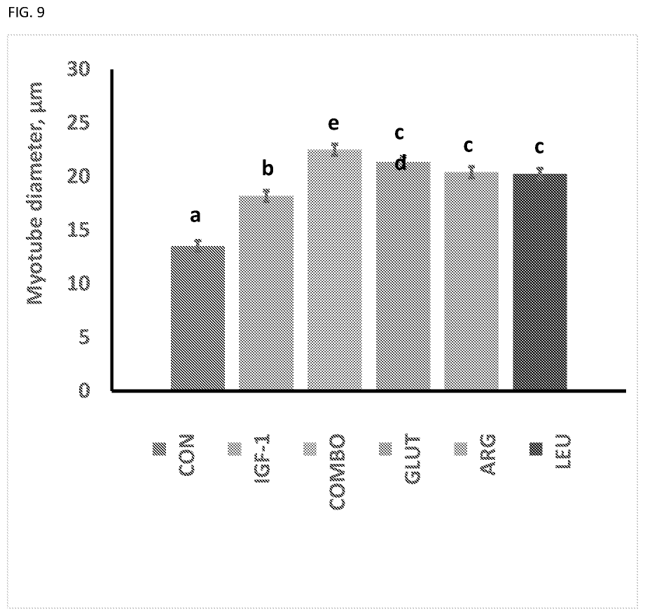

To evaluate cross species efficacy experiments were conducted with avian satellite cells. Avian satellite cells with the control consisting of DM, and treatments containing 5 ng/mL yeast produced IGF-1, a combination of 5 ng/mL yeast produced IGF-1, L-leucine (1 mM), L-arginine (1 mM), L-glutamine (3 mM), and each amino acid individually were evaluated by adding them to DM, data shown in FIG. 9.

Results

As outlined in FIG. 1, active IGF-1 and individual amino acids were able to increase the myotube size compared to the control treatment, with L-glutamine, L-leucine, and L-arginine having a more profound growth response of myotubes compared to L-methionine, or L-lysine. This finding led to the next experiment where yeast produced active IGF-1, L-glutamine, L-leucine, and L-arginine were applied to satellite cells in combination. This specific combination of active IGF-1 and amino acids resulted in an additive response in myotube growth.

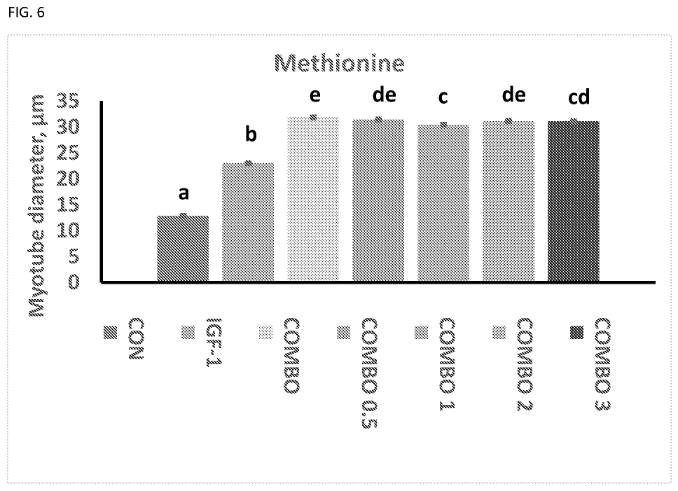

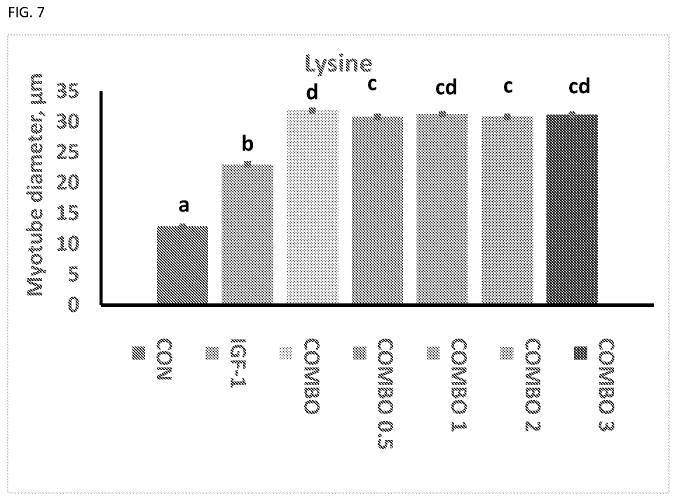

The next series of experiments was set out to determine the useful inclusion of each individual amino acid. The useful inclusion of each amino acid is the minimum amount of each required to produce the maximal growth response. The data collected that is outlined in FIGS. 3-5, the blend of amino acids was 3 mM of L-glutamine, 1 mM L-leucine, and 1 mM L-arginine. To confirm these amino acids are the useful combination of amino acids experiments were conducted by titrating L-methionine and L-lysine. As illustrated in FIGS. 6 and 7, the addition of L-methionine and L-lysine did not provide any increase in myotube growth. Finally, we hypothesized that the mode of action of the unique combination of amino acids was by triggering mTOR pathway signaling, and this was substantiated since when rapamycin was added treated SC had a similar myotube size as control cultures (FIG. 8).

The evaluation of the cross-species efficacy was confirmed by experimentation utilizing avian satellite cells. The data outlined in FIG. 9 show that each component that was effective in improving porcine satellite cell growth was also effective in improving avian satellite cell growth. Additionally, the specific combination of 5 ng/mL yeast produced active IGF-1, 3 mM L-glutamin, 1 mM L-leucine, and 1 mM L-arginine provided a useful cellular response. The previous mechanistic research that employed satellite cells as a model for animal growth suggests that the combinatorial inclusion of active IGF-1, L-glutamine, L-leucine, and L-arginine will be effective in promoting growth when used as a feed supplement when fed to pigs and chickens (Shappell et al., 2000; Vaughn et al., 2017).

### CITATIONS FOR EXAMPLE 1

Shappell, N. W., V. J. Feil, D. J. Smith, G. L. Larsen, and D. C. McFarland. 2000. Response of C2C12 mouse and turkey skeletal muscle cells to the beta-adrenergic agonist ractopamine. Journal of animal science 78(3):699-708.
Vaughn, M., K. Phelps, and J. Gonzalez. 2017. In vitro supplementation with the porcine plasma product, betaGRO®, stimulates activity of porcine fetal myoblasts and neonatal satellite cells in a divergent manner. Animal: an international journal of animal bioscience: 1-9.

### Example 2

Evaluation of Supplement on Broiler Performance

A 42-day feeding trial was carried out in a commercial research institution to determine the efficacy of the supplement (CT). The supplement CT contained 450 ng/g of yeast derived active IGF-1, 93 wt % yeast, 4.4 wt % L-glutamine, 1.7 wt % L-arginine, and 1.3 wt % L-leucine. The birds were grown under two different environments. One was a “Low Stress” environment using clean wood shavings litter, while the other was a “High Stress” environment, in which used wood shavings were obtained from a commercial farm that had suffered high mortality; the shavings were also seeded with coccidia oocysts and Clostridia bacteria to simulate a commercial environmental challenge. The supplement was fed at the rate of 300 g CT/ton in starter chicken feed (0-14 d) and 50 g CT/ton in grower chicken feed (14-28 d) and 50 g CT/ton in finisher chicken feed (28-42 d) diets. Another group of birds in the low stress environment was fed 300 g supplement/ton only in the starter chicken feed.

Growing birds in the high stress environment had a negative impact upon overall bird performance; however, the addition of CT successfully mitigated the detriments of the high stress environment and in many cases performance was equal to or better than the performance of birds on the negative control diet (the complete diet without the supplement) in the low stress environment. Significant improvements in 42-day body weight and feed conversion were observed in chicks grown in both environments due to the addition of CT. Birds fed CT were more uniform than birds grown on the respective negative control diets, a factor important in processing. Mortality of birds on the severe stress environment was significantly reduced by the addition of CT. Addition of CT resulted in significant improvements in total carcass yield and breast meat yield, expressed both as a percentage of carcass weight and in total amount produced. This was observed under both environmental conditions.

### Description of Problem

The period after hatch of broilers is critical for development and adaptation of the small intestine to nutritional changes (Whitten et al., 2012). Additionally, during the post-hatch period the broilers immune system is naïve and skeletal muscle has its greatest metabolic demand. Post-hatch nutrition has potential to program life-long immune and muscle metabolism. Restriction of energy and amino acid density during the first 14 days hindered production performance and exacerbated muscle myopathies (Meloche et al., 2018), thus implying the importance of the first 14 days of life on skeletal muscle development. Additionally, post-hatch feed restrictions result in depressing myogenic signaling regulatory pathways (Velleman et al., 2010; Velleman et al., 2014). For many years, spray-dried plasma (SDP) proteins have been widely used in diets for nursery pigs. The SDP contains functional proteins including immunoglobulins and biologically active peptides, that may play crucial roles in cellular signaling. Recently, studies have evaluated the use of SDP in poultry diets with positive results (Campbell et al., 2003; Bregendahl et al., 2005; Jamroz et al., 2012; Henn et al., 2013). These positive results include greater body weight gain, with a pronounced benefit during exposure to pathogenic bacteria (King et al., 2005; Beski et al., 2015; Beski et al., 2016).

Materials and Methods

A study was conducted to evaluate the performance of broiler chickens fed diets with the supplement (CT). The supplement CT is manufactured to contain 450 ng/g of yeast derived IGF-1, 93% yeast, 4.4% L-glutamine, 1.7% L-arginine, and 1.3% L-leucine. The trial was carried out in a well-recognized commercial testing laboratory in the United States to simulate a commercial environment.

The products were evaluated under two environmental scenarios: a low-stress environment using clean wood shavings litter, and a severe-stress environment, in which litter was obtained from a commercial farm that had previously suffered high mortality. In addition, clostridia bacteria and coccidia oocysts were scattered in the litter of these pens. CT was fed at the rate of 300 g/ton in starter (0-14 d) and 50 g/ton in grower (14-28 d) and finisher (28-42 d) diets. A common basal diet was used as the carrier for the test articles (Table 1). Nutritionally complete vitamin and trace mineral mixes were used to fortify the diets. A common basal diet was prepared, and aliquots were used in preparing the test diets.

TABLE 1
Composition (%) and nutrient analysis of diets

| | Starter | Grower | Finisher |
|---|---|---|---|
| Ingredient | 0-14 d | 14-28 d | 28-42 d |
| Yellow corn | 45.787 | 51.439 | 55.380 |
| Soybean meal | 38.285 | 34.547 | 30.734 |
| Soybean oil | 8.692 | 8.040 | 8.258 |
| Blended animal protein | 2.000 | 2.000 | 2.000 |
| DL Methionine | 0.439 | 0.260 | 0.190 |
| Salt | 0.599 | 0.549 | 0.499 |
| L-Lysine HCl | 0.088 | 0.034 | 0.042 |
| Limestone | 1.464 | 1.267 | 1.197 |
| Dicalcium phosphate | 2.334 | 1.597 | 1.474 |
| Choline CL 60% | 0.112 | 0.067 | 0.026 |
| Vitamin-Trace minerals | 0.100 | 0.100 | 0.100 |
| Phytase | 0.100 | 0.100 | 0.100 |
| TOTAL | 100.000 | 100.000 | 100.000 |
| Crude protein % | 23.000 | 21.500 | 20.000 |
| ME kcal/lb | 1425.000 | 1450.000 | 1475.000 |
| Lysine % | 1.400 | 1.250 | 1.150 |
| TSAA % | 1.150 | 0.950 | 0.850 |
| Methionine % | 0.788 | 0.599 | 0.513 |
| Calcium % | 1.100 | 0.900 | 0.840 |
| Available P % | 0.600 | 0.450 | 0.420 |
| Total P % | 0.904 | 0.734 | 0.692 |
| Sodium % | 0.260 | 0.240 | 0.220 |

The test period began on Trial Day 0 (day of hatch of chicks) and ended on Trial Day 42. Each pen contained 52 mixed-sex Cobb broilers (50:50 ratio) randomly assigned into 12 replicates per group. Sixty-six chicks were placed in each pen on Day 0 and the number reduced to 52 on Day 1 after crop-fill measurements for individual birds and verifying gender by feather checking. Birds were not replaced for the remainder of the study. The chicks were observed daily for signs of unusual grow-out patterns or health problems. All birds received Coccidiosis vaccine in the hatchery. No antibiotics were administered during the entire trial.

Birds were individually weighed at 1, 7, 14, 28 and 42 days of age and feed consumption determined. Salmonella incidence was tested (two males and two females per pen at 14 days and five males and five females per pen at 42 days) to simulate counts required by USDA/FSIS at processing. Also, at 14 days and 42 days intestinal samples were taken from two males and two females per pen. Samples were taken from two gut areas per bird, one at the distal end of the duodenal loop and the second approximately two inches anterior to Meckel's diverticulum. Lesion scores were determined by the method of Johnson and Reid (1970) and numbers of various organisms determined. Ileum villi height and crypt depth were measured. At 42 days of age ten birds per pen were processed and parts yield determined.

Data were analyzed with SAS 9.4. A Grubb's Test was employed to observe and remove for potential ‘outliers.’ None were found in these studies. Treatment were included as categorical fixed effect. The supplemented groups were compared to the untreated control group as reference. All data sets, for each time period, was analyzed using ordered regression models using SAS Statistical Package. Residual plots were checked to evaluate model fit. Statistical significance was determined at P<0.05.

Results and Discussion

Performance of the broilers was significantly affected overall by the growing environment (Table 2). Almost every production measurement was negatively impacted by the severe stress environment. However, the addition of CT at 300 g/ton in starter diet and 50 g/ton in grower and finisher diets (300-50-50) helped to overcome the performance depression. Body weights were significantly improved by the addition of CT at 7, 14, 28, and 42 days of age. The improvements were especially noted in the group of birds subjected to the severe environmental stress. In birds grown in the low stress environment, at 42 days birds fed the CT were 175 g heavier than those fed the negative control. In birds grown in the severe stress environment, birds fed CT were 262 g heavier than the respective negative control birds. The birds in the severe environment group fed CT were significantly heavier than those fed the negative control in the low stress environment.

TABLE 2
Live performance of birds fed betaGRO and the supplement
under different environmental conditions

| | | Low Stress | | Severe Stress | |
|---|---|---|---|---|---|
| | | Environment² | | Environment³ | |
| | | Neg | cT⁴ | Neg | cT |
| MEASUREMENT¹ | | Control | 300-50-50 | Control | 300-50-50 |
| Body weight (g) | | | | | |
| 7 | d | 168.42^{b} | 190.15^{a} | 169.80^{b} | 191.03^{a} |
| 14 | d | 446.91^{d} | 531.73^{a} | 411.00^{e} | 488.11^{c} |
| 28 | d | 1295.96^{d} | 1460.94^{a} | 1204.55^{e} | 1357.65^{c} |
| 42 | d | 2458.85^{d} | 2634.02^{ab} | 2284.07^{e} | 2546.02^{c} |
| Feed Conversion | | | | | |
| (feed/gain) | | | | | |
| 0-7 | d | 0.934^{b} | 0.873^{a} | 0.915^{b} | 0.883^{a} |
| 0-14 | d | 1.114^{b} | 1.054^{a} | 1.156^{c} | 1.067^{a} |
| 0-28 | d | 1.401^{c} | 1.350^{a} | 1.467^{d} | 1.409^{c} |
| 0-42 | d | 1.844^{c} | 1.770^{a} | 1.950^{d} | 1.825^{bc} |
| Mortality % | | | | | |
| 0-7 | d | 0.481^{a} | 0.481^{a} | 0.481^{a} | 0.321^{a} |
| 0-14 | d | 0.962^{a} | 0.641^{a} | 3.846^{c} | 2.404^{b} |
| 0-28 | d | 0.962^{a} | 0.641^{a} | 3.846^{c} | 2.404^{b} |
| 0-42 | d | 1.563^{a} | 1.215^{a} | 8.333^{c} | 4.688^{b} |
| Body weight | | | | | |
| uniformity (% CV) | | | | | |
| 0-7 | d | 14.73^{b} | 10.18^{a} | 14.96^{b} | 10.35^{a} |
| 0-14 | d | 18.18^{c} | 12.59^{a} | 20.52^{d} | 13.61^{b} |
| 0-28 | d | 15.38^{d} | 9.90^{a} | 18.39^{e} | 11.00^{bc} |
| 0-42 | d | 12.03^{b} | 10.24^{a} | 16.95^{c} | 10.94^{a} |
| European Production | | | | | |
| Efficiency Factor⁵ | | | | | |
| 0-7 | d | 255.46^{b} | 309.08^{a} | 263.10^{b} | 307.76^{a} |
| 0-14 | d | 282.85^{c} | 357.66^{a} | 239.64^{d} | 316.13^{b} |
| 0-28 | d | 315.30^{d} | 372.14^{a} | 261.48^{e} | 319.01^{d} |
| 0-42 | d | 307.24^{c} | 344.63^{a} | 245.91^{d} | 309.09^{c} |

¹Twelve replicate pens of 52 mixed-sex birds per treatment.
²Clean wood shavings litter.
³Wood shavings from a house with high incidence of mortality and overseeded with Clostridium spores and coccidial oocysts.
⁴the supplement.
⁵EPEF = (Average grams gained/day × % survival rate)/(Feed conversion × 10)
^{abc}Means in row with common superscript do not differ significantly (P < 0.05).

Similar responses were observed for feed conversion. Birds grown under the severe stress environment had significantly poorer feed conversion than cohort birds grown under the low stress environment. However, the addition of CT to the diet at 300 g/ton in starter diet and 50 g/ton in grower and finisher diets significantly improved feed conversion in both environmental scenarios. Birds fed CT in the low stress environment had 0.07 points lower 0-42 day feed conversion than those fed the negative control. Birds fed CT in the severe stress environment had 0.14 and 0.13 points lower feed conversion at 42 days compared to those fed the negative control. Birds fed the diets with CT in the severe environment did not differ significantly from those fed the negative control under the low stress environment.

Mortality was severely influenced by the environment in which the birds were grown. Birds grown under the low stress environment suffered minimal mortality with no significant difference among birds on the different dietary treatments. However, birds grown under the severe stress environment suffered heavy mortality, as might be expected. The addition of CT significantly reduced mortality in birds grown in this environment by nearly half as compared to those fed the negative control diet.

Flock uniformity, a factor that is important during processing, was significantly improved in both environments. Birds fed CT had significantly better flock uniformity than those fed the respective negative controls. The severe environmental stress conditions decreased flock uniformity of those fed the negative control diet but did not significantly affect those fed diets with CT.

The European Production Efficiency Factor (EPEF) is a formula that considers body weight gain, feed conversion, and mortality to result in a single value that gives an overall picture of performance. Because of better gain, improved feed conversion, and reduced mortality, birds fed the diets with CT had significantly higher EPEF values than those fed the negative control diets under both environmental conditions.

Economically important processing factors were significantly influenced by both the environmental conditions under which the birds were grown and by the inclusion of CT (Table 3). For birds grown under the low stress conditions, those fed CT had 0.95% higher chilled carcass yield, compared to birds fed the control diet. Birds fed the negative control diets in the severe stress environment had 2.93% lower chilled carcass yield than those fed the negative control diets in the low stress environment. However, birds fed the diets CT under the high stress conditions had chilled carcass yields that were similar to those of birds fed CT under the low stress conditions, and were 4.45% higher than those fed the negative control diet under the severe environmental conditions.

TABLE 3
Forty-two-day processing yields of birds fed betaGRO and
the supplement under different environmental conditions.

| | Low Stress | | Severe Stress | |
|---|---|---|---|---|
| | Environment¹ | | Environment² | |
| | Negative | cT⁴ | Negative | cT |
| MEASUREMENT³ | Control | 300-50-50 | Control | 300-50-50 |
| Carcass Yield | 73.99^{b} | 74.94^{a} | 71.07^{c} | 75.52^{a} |
| (Chilled) | | | | |
| Pectoralis major | 20.32^{c} | 21.07^{a} | 19.08^{d} | 20.53^{bc} |
| (% post-chill) | | | | |
| Pectoralis major (g) | 388.79^{d} | 434.58^{a} | 327.02^{e} | 412.84^{bc} |
| Pectoralis Minor | 5.07^{c} | 5.87^{a} | 4.76^{d} | 5.19^{c} |
| (% post-chill) | | | | |
| Pectoralis minor (g) | 3.767^{e} | 4.430^{a} | 3.395^{f} | 3.937^{cd} |
| Whole Breast | 25.39^{c} | 26.94^{a} | 23.84^{d} | 25.72^{bc} |
| (% post-chill) | | | | |
| Whole Breast (g) | 392.56^{d} | 439.01^{a} | 330.42^{e} | 416.78^{bc} |
| Thigh Yield | 14.67^{b} | 15.21^{ab} | 13.53^{c} | 14.74^{b} |
| (% post-chill) | | | | |
| Wing Yield | 10.65^{b} | 11.40^{a} | 9.74^{c} | 10.62^{b} |
| (% post-chill) | | | | |
| Leg Yield | 13.48^{c} | 14.31^{a} | 12.33^{d} | 13.51^{bc} |
| (% post-chill) | | | | |
| Abdominal Fat | 1.39^{c} | 1.47^{ab} | 1.30^{d} | 1.41^{bc} |
| (% post-chill) | | | | |

¹Clean wood shavings litter.
²Wood shavings from a house with high incidence of mortality and overseeded with Clostridium spores and coccidial oocysts.
³Twelve replicates of ten birds per treatment.
⁴ the supplement.
^{abc}Means in row with common superscript do not differ significantly (P < 0.05).

Total breast meat yield was also significantly affected by both the environmental conditions under which the birds were raised. When grown under low stress conditions, birds fed CT had 1.39% greater whole breast yield as a percentage of live weight. When grown under the high stress conditions, birds fed the negative control diet had 1.84% less whole breast meat than those fed the same diet under low stress conditions. Birds fed diets with CT under the severe stress conditions had 2.47% higher whole breast yield than those fed the negative control diet and were significantly higher yield than those fed the negative control diet under low stress conditions.

If one examines the important breast marketing factors, addition of CT at 300 g/ton in starter diet and 50 g/ton in grower and finisher diets resulted in significantly more pectoralis major, pectoralis minor, and total breast yield than did those fed the negative control, under either environmental conditions. In fact, the quantity of breast meat of broilers fed CT under the severe stress environment was equal to or greater than that of birds fed the negative control in the low stress environment.

For birds grown under the low stress environment there was no significant difference in villi development intestinal lesion scores among birds fed the negative control diet or the diets with CT (Table 4). However, under the severe stress environment, birds fed CT had significantly greater villi cell height and crypt depth than those fed the negative control. There was a significant difference in lesion scores between birds fed under the low stress environment and the severe stress environment, as would be expected. In birds fed under the severe stress routine, supplementation with CT had significantly lower lesion scores than those fed the negative control diet. These improvements should aid in better digestibility of the dietary nutrients.

TABLE 4
Intestinal measurements of birds fed betaGRO and the
supplement under different environmental conditions

| | Low Stress | | Severe Stress | |
|---|---|---|---|---|
| | Environment | | Environment | |
| | Negative | cT | Negative | cT |
| Measurement | Control | 300-50-50 | Control | 300-50-50 |
| Lesion Scores 14 d | 0.27^{a} | 0.29^{a} | 1.69^{c} | 0.92^{b} |
| Lesion Scores 42 d | 0.29^{a} | 0.24^{a} | 1.61^{c} | 0.60^{b} |
| Villi Cell Height | 985.50^{a} | 1024.77^{a} | 926.79^{b} | 983.31^{a} |
| (μm) 14 d | | | | |
| Crypt Depth (μm) 14 d | 432.56^{b} | 469.27^{ab} | 375.65^{c} | 450.77^{ab} |
| Villi Height: Crypt | 2.37^{a} | 2.26^{a} | 2.64^{b} | 2.28^{a} |
| Depth Ratio | | | | |
| E. coli (log₁₀) 14 d | 4.85^{a} | 5.14^{a} | 6.73^{c} | 6.04^{b} |
| E. coli (log₁₀) 42 d | 4.92^{a} | 5.12^{a} | 6.67^{c} | 6.04^{b} |
| APC (log₁₀) 14 d | 8.09^{a} | 8.17^{a} | 8.34^{a} | 8.00^{a} |
| APC (log₁₀) 42 d | 8.11^{a} | 8.14^{a} | 8.13^{a} | 8.09^{a} |
| Clostridium perfringens | 2.87^{a} | 2.78^{a} | 4.34^{d} | 3.16^{b} |
| (log₁₀) 14 d | | | | |
| Clostridium perfringens | 2.92^{ab} | 2.77^{a} | 4.25^{c} | 3.39^{b} |
| (log₁₀) 42 d | | | | |
| Salmonella | 20.83^{a} | 22.92^{a} | 81.25^{c} | 62.50^{b} |
| Incidence (%) 14 d | | | | |
| Salmonella | 15.83^{a} | 10.00^{a} | 75.83^{c} | 46.67^{b} |
| Incidence (%) 42 d | | | | |
| Oocyst/g feces 14 d | 6.02^{a} | 6.18^{a} | 6.15^{a} | 6.05^{a} |
| Oocyst/g feces 42 d | 6.16^{a} | 6.15^{a} | 6.08^{a} | 6.13^{a} |

^{abcd}Means in row with common superscript do not differ significantly (P < 0.05).

Additionally, birds fed under the severe stress environment had greater numbers of E. coli at 14 and 42 days than those fed under the low stress environment, with significant reductions in birds fed CT. The incidence of Salmonella was higher in birds fed under the severe stress environment, with significantly lower numbers at both 14 and 42 days of age when fed diets with CT. The incidence of Clostridia organisms, strongly tied to necrotic enteritis, was significantly higher in birds fed under the severe stress environment but were significantly reduced in birds fed CT. Neither diets or environment had a significant effect on antigen presenting cells (APC) or oocyst count.

The improvements in body weight and feed conversion noted from the addition of CT agree with the results of Campbell et al. (2003), Bregendahl et al. (2005), Jamroz et al. (2012), and Henn et al. (2013) who fed diets containing porcine or bovine plasma protein. The greater response to CT under challenge conditions agrees with the findings of Henn et al., (2013). The improvements seen in gut morphology agree with the results of King et al. (2005) and Beski et al. (2015). The improvement in performance of birds fed CT during exposure to highly pathogenic bacteria agrees with the work of Beski et al. (2016). Collectively, these comparisons point out the unique and unexpected nature of CT as a non-animal derived product exerting biological effects previously associated with feeding animal-derived mixtures of proteins.

Many of the responses observed in the current research to the supplementation CT are consistent with data previously reported when broilers were supplemented with spray-dried plasma (SDP) (Campbell et al., 2003; Bregendahl et al., 2005; Kind et al., 2005; Jamroz et al., 2012; Henn et al., 2013; Beski et al., 2015; Beski et al., 2016); however, the inclusion rate of CT into the diets was significantly lower; only 1.5-6 percent of the inclusion rate of SDP. Therefore, the response to CT supplementation is likely not nutritive but due to metabolism improvements. Recent research has demonstrated that betaGRO (BG), an animal protein derived product fed at a similar rate as CT, promoted immune cell function and skeletal muscle growth in vitro (Vaughn et al. 2017; Vaughn et al. 2018). Inclusion of BG in culture medium at 10 mg/mL resulted in significantly larger myotube size and was mediated by positive changes in Mechanistic Target of Rapamycin (mTOR) signaling proteins (Vaughn et al., 2017). Immune research conducted by Vaughn et al. (2018) observed increased respiratory metabolism of actively growing β-lymphocyte cells by more than two-fold, which was mediated by mTOR pathway signaling since the addition of rapamycin abolished all positive treatment effects. This in conjunction with the current data suggest that supplementation of CT in a severe stress environment bolstered the broilers immune system to mitigate bacterial interference on the small intestine that result in performance detriments, while simultaneously promoting superior lean tissue growth.

### Conclusions and Applications

The addition of CT, a yeast-based product, at the rate of 300 g/ton in diets fed 0-14 days followed by 50 g/ton to 42 days, significantly improved growth rate, feed conversion, livability, dressing percentage, and breast meat yield of broilers.

Birds grown under severe stress environment responded to a greater extent to the addition of CT than those fed in a lower-stress environment.

Improvements in villus height and crypt depth in birds fed CT may be related to the improvements in performance.

Birds fed CT had lower incidence of carcass Salmonella, fewer intestinal bacteria, and lower lesion scores than those fed the negative control diets.

The low inclusion rate of CT, suggest that performance improvements are driven by mechanistic metabolism improvements, resulting in healthier broilers with greater growth rates.

### CITATIONS FOR EXAMPLE 2

Beski, S. S. M., R. A. Swick, and P. A. Iji. 2015. Subsequent growth performance and digestive physiology of broilers fed on starter diets containing spray-dried porcine plasma as a substitute for meat meal. Br. Poult. Sci. 56:559-568.
Beski, S. S. M., R. A. Swick, and P. A. Iji. 2016. Effect of dietary inclusion of spray-dried porcine plasma on performance, some physiological and immunological response of broiler chickens challenged with salmonella. J. Anim. Physiol. Anim. Nutr. 100:957-966.
Bregendahl, K D. U. Ahn, D. W. Trampel, and J. M. Campbell. 2005. Effects of dietary spray-dried bovine plasma protein on broiler growth performance and breast-meat yield. J. Appl. Poult. Res. 14:560-568.
Campbell, J. M., J. Quigley, L. Russel, and M. Kidd. 2003. Effect of spray-dried bovine serum on intake, health, and growth of broilers housed in different environments. J. Anim. Sci. 81:2776-2782.
Henn, J. D., J. Bockor, M. S. Viera, A. M. L. Riberio, A. M. Kessler, L. Albino, H. Rostagno, J. D. Crenshaw, J. M. Campbell, and L. F. S. Rangel. 2013. Inclusion of porcine spray-dried plasma in broiler diets. J. Appl. Poult. Res. 22:229-237.
Jamroz, D., A. Wiliczkiewicz, J. Orda, J. Kuryszko, and T. Stefaniak. 2012. Use of spray-dried porcine blood by-products in diets for young chickens. J. Anim. Physiol. Anim. Nutr. 96:319-333.
Johnson, J., and M. Reid. 1970. Anticoccidal drugs: lesion scoring techniques in battery and floor pen experiments with chickens. Exp. Parasitol. 28:30-36.
King, M. R., V. Ravindran, P. C. H. Morel, D. V. Thomas, M. J. Birtles, and J. R. Pluske, and L. Bocktor. 2005. Effects of spray-dried colostrum and plasma on the performance and gut morphology of broiler chicks. Aust. J. Agric. Res. 56:811-817.
Meloche, K., B. Fancher, D. Emmerson, S. Bilgili, and W. Dozier III. 2018. Effects of reduced dietary energy and amino acid density on Pectoralis major myopathies in broiler chickens at 36 and 49 days of agel. Poult. Sci. 97(5):1794-1807.
Vaughn, M., K. Phelps, and J. Gonzalez. 2017. In vitro supplementation with the porcine plasma product, betaGRO®, stimulates activity of porcine fetal myoblasts and neonatal satellite cells in a divergent manner. Animal.: an international journal of animal bioscience:1-9.
Vaughn, M., M. Rahe, J. Loughmiller, M. Murtaugh. 2018. Targeting immune cell energetics to produce healthy pigs. National hog farmer, Industry voice. https://www.nationalhogfarmer.com/animal-health/targeting-immune-cell-energetics-produce-healthy-pigs.
Velleman, S., C. Coy, and D. Emmerson. 2014. Effect of the timing of posthatch feed restrictions on broiler breast muscle development and muscle transcriptional regulatory factor gene expression. Poult. Sci. 93(6):1484-1494.
Velleman, S. G., K. Nestor, C. Coy, I. Harford, and N. Anthony. 2010. Effect of posthatch feed restriction on broiler breast muscle development and muscle transcriptional regulatory factor gene and heparan sulfate proteoglycan expression. Int. J. Poult. Sci 9:417-425.
Whitten, P. J. A., D. J. Langhout, and M. W. A. Verstegen. 2012. Small intestine development in chicks after hatch and in pigs around the time of weaning in relation to nutrition: a review. Acta. Agric. Scand. Section A, Vol. 62, pp 1-12.

### Example 3

Evaluation of Supplement on Piglet Performance

Materials and Methods

The feed supplement described in Example 2 was evaluated on its ability to influence growth performance, feed efficiency, and gut health in nursery pigs. A 42-day trial was conducted at a commercial research facility. Diets were formulated in three phases to best match piglet nutritional needs. Piglets were weaned at 19 days of age, which is considered trial day 0, phase 1 diets were fed from day 0-7, phase 2 diets were fed from day 8-21, and phase 3 diets were fed from day 22-42 of the trial. The control diet did not contain any additives to the base diet (CON), a positive control that contained betaGRO® at 2.5 kg per ton in phase 1, and 1.5 kg per ton in phase 2 (BG), a treatment that contained the supplement at 2.5 kg per ton in phase 1, and 1.5 kg per ton in phase 2 (CT1), a treatment that contained the supplement at 350 g per ton in all three phases (CT2), and a treatment that contained the supplement at 175 g per ton in all three phases (CT3). Response criteria contained body weight gain, feed conversion, and fecal scores.

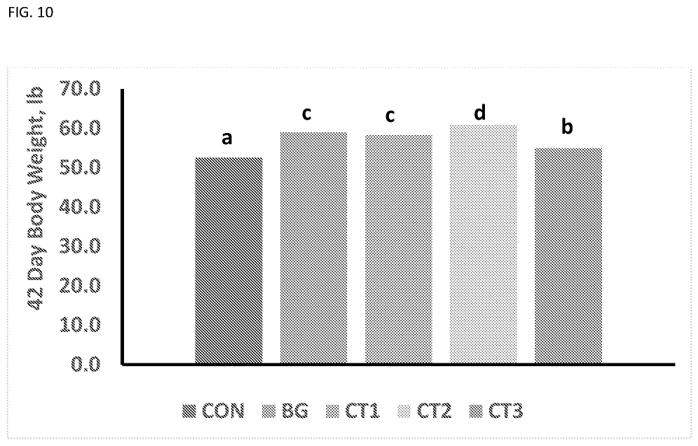

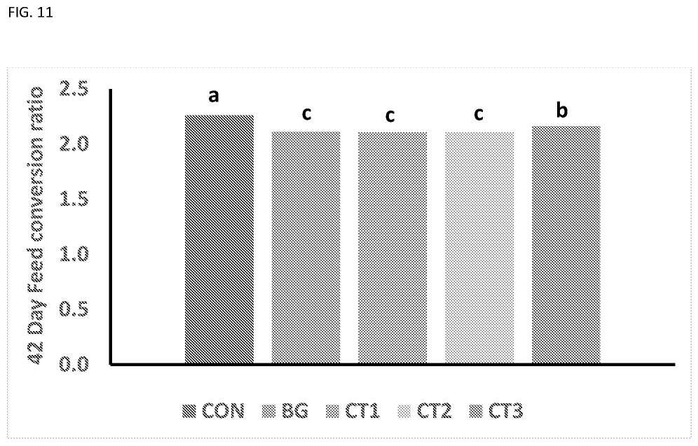

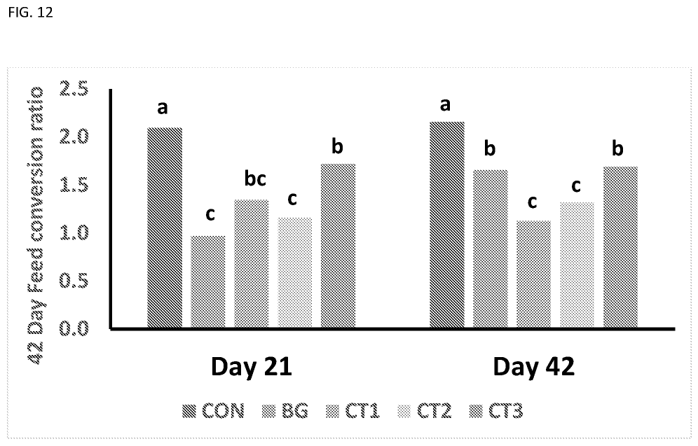

Results (FIGS. 10-12)

The addition of betGRO and the supplement resulted in greater body weight at day 42 of the trial than the control (P<0.05). Pigs within the CT2 treatment had the greatest (P<0.05) day 42 body weight of all treatments. Pigs in the BG and CT1 treatment had a similar day 42 bodyweight and greater day 42 body weight compared to CT3 and the CON (P<0.05). The CT3 had a greater day 42 body weight compared to the CON treatment.

The addition of betaGRO and the supplement to the diet resulted in an improved feed conversion ratio (P<0.05). The pigs fed the BG, CT1, and CT2 had a better feed conversion compared to CT3 (P<0.05), and CT3 had better feed conversion compared to the CON. As an indication for gut health fecal diarrhea scores were collected.

At both day 21 and day 42 of the trial piglets fed betaGRO or the supplement had decreased fecal diarrhea scores compared to the CON (P<0.05). At day 21 of the trial the fecal scores from BG and CT2 had lower fecal scores compared to CT3 (P<0.05), while CT1 fecal scores were intermediate (P>0.05). At day 42 of the trial piglets from CT1 and CT2 had lower fecal scores compared to all other treatments (P<0.05), and BG and CT3 had lower fecal scores than CON (P<0.05).

### Example 4

Evaluation of Supplement on Skeletomuscular Driven Growth in Avian Species

Materials and Methods

The feed supplement described in Example 2 was evaluated on its ability to influence growth performance, feed efficiency, and gut health in turkey poults. An 84-day trial was conducted at a commercial research facility. Diets were formulated in three phases to best match nutritional needs of the turkeys.

The starter diets were fed from day 0-28, grower diets were fed days 29-56, and finisher diets were fed days 57-84 of the trial.

The control diet did not contain any additives to the base diet (CON), a treatment that contained the supplement at 300 g per ton in the starter, and 50 g per ton in phase 2 (CT1), a treatment that contained the supplement at 600 g per ton in the starter, and 100 g per ton in phase 2 (CT2), a treatment that contained the supplement at 600 g per ton in the starter, and 50 g per ton in phase 2 (CT3). Response criteria contained body weight gain, feed conversion, gut health, and intestinal bacteria prevalence.

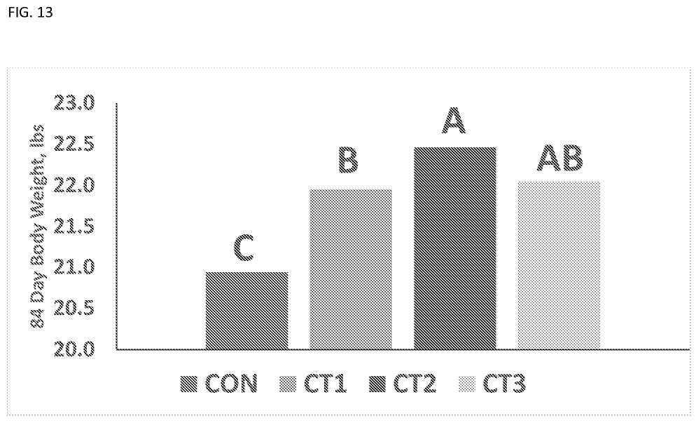

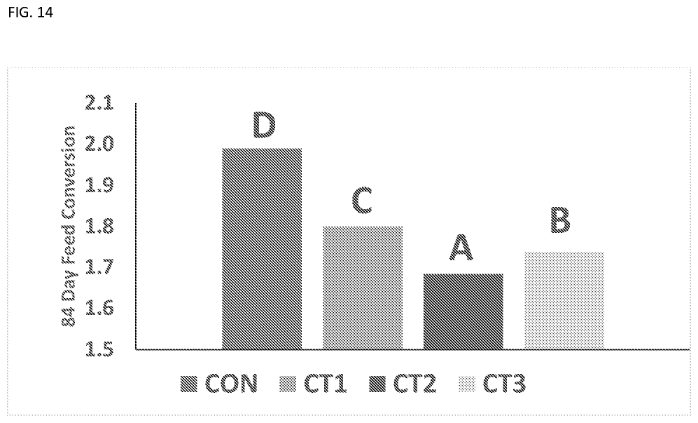

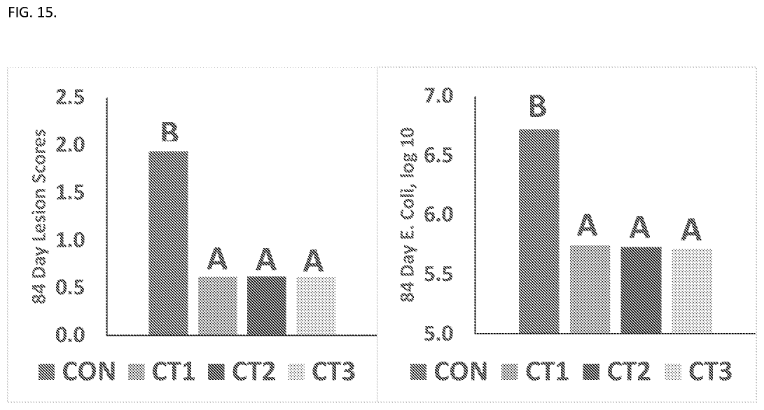

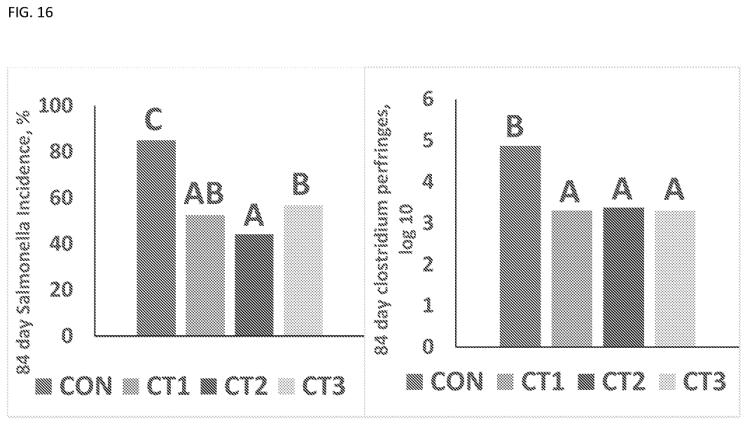

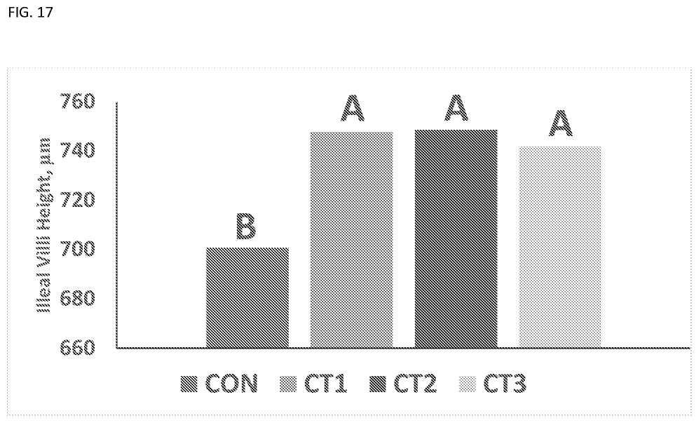

Results (FIGS. 13-17)

When the supplement was administered to turkey poults body weight was increased and feed conversion was improved with all supplement doses compared to the CON (P<0.05). Turkeys in the CT2 treatment had the greatest body weight of all treatments (P<0.05), while turkeys fed CT1 and CT3 had similar body weights (P>0.05), which were greater than the CON turkeys body weights (P<0.05). Additionally, CT2 had a better feed conversion ratio compared to CT1 and CT3 (P<0.05), and CT3 had a better feed conversion than CT1 (P<0.05).

On an objective scale of intestinal lesions, the turkeys fed all dosages of the supplement had lower lesion scores compared to turkeys in the CON treatment (P<0.05), and all supplement treatments had similar lesion scores (P>0.05). When E. coli presence in the small intestine was measured the turkeys among all supplement treatments had a decreased prevalence of E. coli compared to the CON fed turkeys (P<0.05). The percentage of turkeys with salmonella present in the small intestine was less for all supplement treatments compared to the CON treatment (P<0.05). Turkeys fed the CT2 dose had a decreased percentage of turkeys with salmonella compared to CT3 turkeys (P<0.05), where the percentage of CT1 turkeys with salmonella was similar to CT2 and CT3 (P>0.05). The log formation of Clostridium perfringes was lower for turkeys fed all dosages of the supplement compared to turkeys in the CON treatment (P<0.05), with all supplement dosages were similar (P>0.05). The illeal villi height was measured and all dosages of the supplement resulted in a greater illeal villi height compared to the CON treatment (P<0.05).

### Example 6

Evaluation of the Supplement on Seabass Performance

Materials and Methods

The supplement described in Example 2 was evaluated on its ability to affect the growth performance, immunity, and health in Asian seabass (Lates calcalifer). A 12-week study was conducted at a university research facility. There was a control with no additives to a complete basal diet, and three treatments that had the supplement added to the basal diet at a of 0.000050, 0.00015, and 0.00045 g/ton of feed. Response criteria included growth rate, feed consumption, feed efficiency, immunity, and gut health.

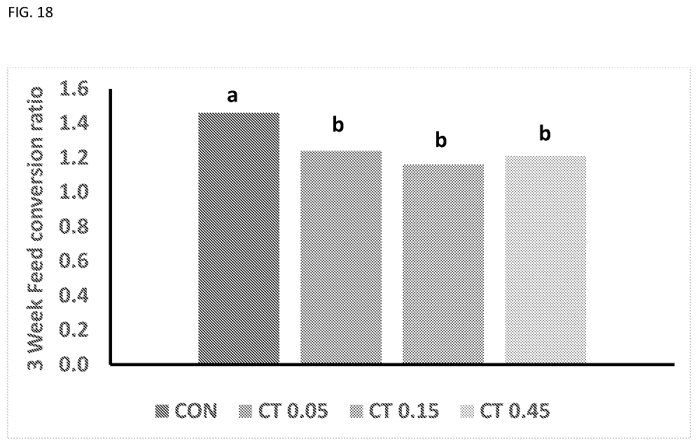

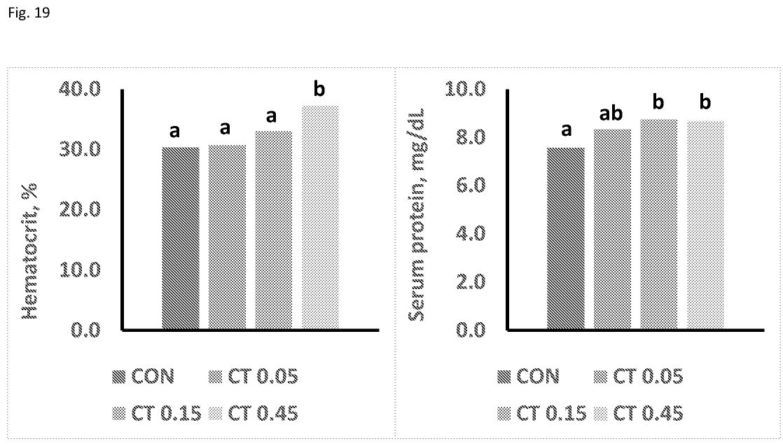

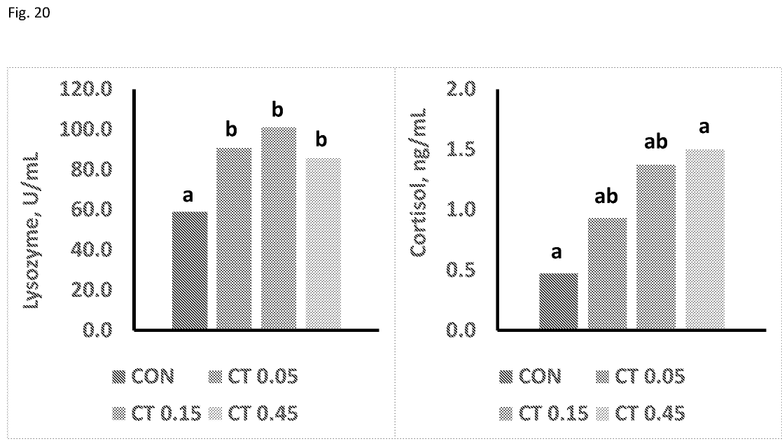

Results (FIGS. 18-20)

When the supplement was fed to seabass there was a significant improvement in feed conversion during the first 3 weeks of the trial (P<0.05), resulting in more efficient growth. The supplement also mounted an immune response, as indicated by a greater hematocrit level at a supplemention of 0.00045 g/ton (P<0.05), and there was a greater concentration of serum protein when the supplement was added at 0.00015 and 0.00045 g/ton of the basal diet (P<0.05). All levels of supplementation resulted in greater lysozyme concentrations (P<0.05), indicating the gut health of the seabass was improved by supplementation. Serum cortisol concentrations indicate the ability to respond to systemic stress, and when supplemented at 0.00045 g/ton cortisol levels were increased (P<0.05) compared to the control. The supplementation of 0.00005 and 0.00015 had similar cortisol concentrations compared to the other treatments (P>0.05).

### Example 6

Evaluation of Supplement on Sow Gestation and Newborn Live Performance

Numerous test products are fed almost routinely today in modern-day sow and piglet production to aid sow farrowing period and improve piglet quality, especially through weaning. Because live performance and gut intestinal health directly affects piglet body weight uniformity, we determined if piglet body weights in the nursery are affected when the supplement is administered.

Materials and Methods

A trial was conducted at a commercial research facility, with 20 sows per treatment and 160 piglets per treatment were tracked for performance through the nursery. The supplement described in Example 2 was used.

The control diet did not contain any additives to the base diet (CON), a diet that contained the supplement at 1000 ppm or 1-kg per metric ton of feed (CT1), a diet that contained the supplement at 350 ppm or 0.35-kg per metric ton of feed (CT2), and a diet that contained the supplement at 100 ppm or 0.10-kg per metric ton of feed (CT3). There was a 7-10-day acclimation period prior to sow estrus, and trial day 0 was the time of estrus. The piglets were fed a common diet to match the piglet's nutritional needs in the nursery and finishing phase.

Test parameter criteria for sow were body condition score and wean to estrus interval. Response criteria for piglets included average daily gain, 126-day body weight, and feed conversion.

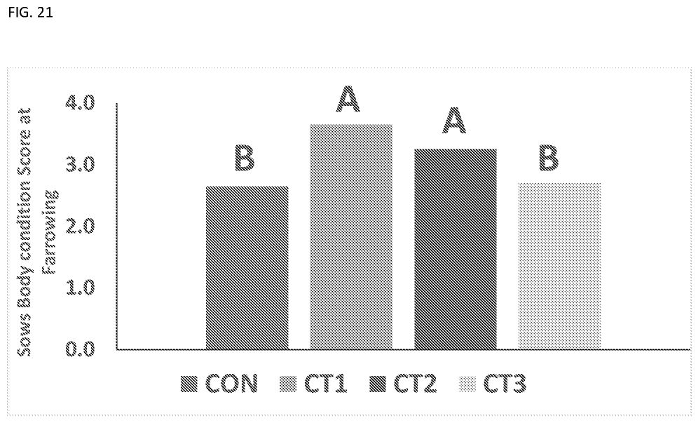

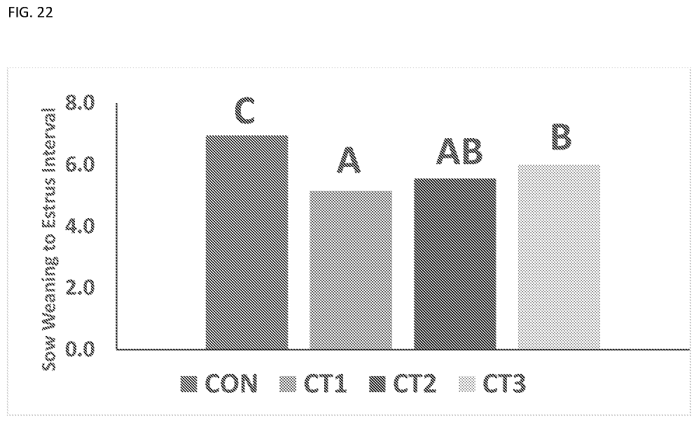

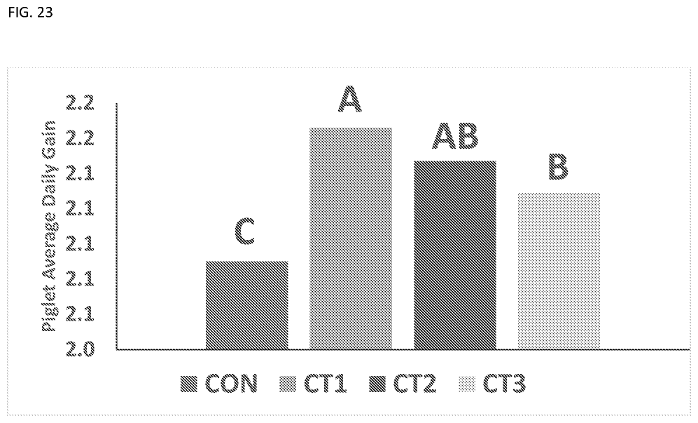

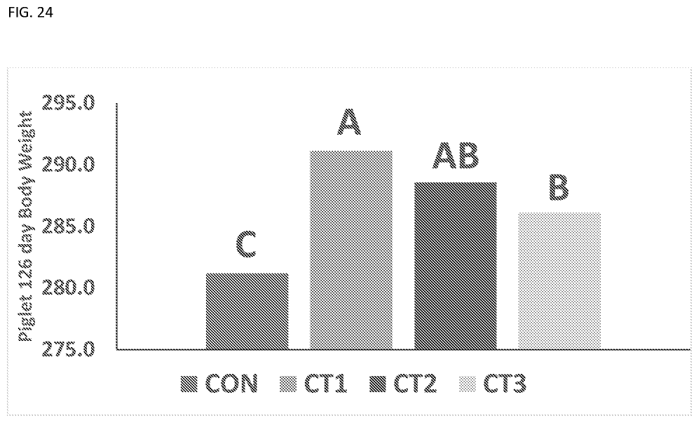

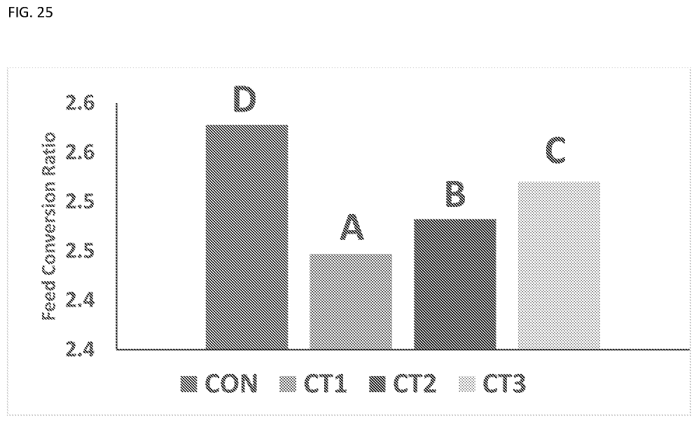

Results (FIGS. 21-25)

Sows responded to supplementation where CT1 and CT2 had greater body condition scores at the end of farrowing compared to CT3 and the CON (P<0.05). Additionally, all supplement-treated sows had a decreased weaning to estrus interval compared to CON sows (P<0.05). Sows in the CT1 treatment had a shorter weaning to estrus interval compared to sows in the CT3 treatment (P<0.05), and sows in the CT2 had a similar weaning to estrus interval as sows in the CT1 and CT3 treatments (P>0.05).

Over the course of the 126 day feeding period piglets reared from sows fed the supplement had greater average daily gain compared to piglets reared from CON sows (P<0.05). Piglets from CT1 sows had a greater average daily gain than piglets from CT3 sows (P<0.05), although piglets from CT2 sows had a similar average daily gain compared to piglets from CT1 and CT3 sows (P>0.05). Additionally, 126-day body weight of piglets reared from sows fed the supplement was greater compared to piglets reared from CON sows (P<0.05). Piglets from CT1 sows had a greater 126 day body weight than piglets from CT3 sows (P<0.05), although piglets from CT2 sows had a similar 126 day body weight compared to piglets from CT1 and CT3 sows (P>0.05). Piglets from CT1 sows had the best feed conversion ratio compared to all other treatments over the course 126 day feeding period (P>0.05). Piglets from CT2 sows had an improved feed conversion ratio compared to piglets from CT3 and CON sows (P>0.05), and piglets from CT3 sows had a better feed conversion ratio compared to piglets from CON sows (P>0.05).

### Example 7

Evaluation of Supplement on Layer Egg Production, Growth Performance, and Feed Efficiency

Materials and Methods

The supplement described in Example 2 was evaluated on its ability to influence egg production, growth performance, and feed efficiency in egg laying hens. A 140-day trial was conducted at a commercial research facility. Each treatment (or experimental) group contained three commercial-type laying hens randomly assigned into 20 replicates per group containing 60 commercial-type laying hens per replicate for a total of 120 animals on study. Trial period began on Day 0 with randomly assigned egg-laying hens at age of 18 weeks of age. Trial completed on Day 140 while fed a consistent industry standard diet during the entire trial period to best meet the laying hen's nutritional needs. The control diet did not contain any additives to the base diet (CON), a treatment diet contained the supplement at 60 grams per ton (CT). Response criteria contained hen body weight, fat pad (% of body weight) of hens, hen intestinal lesion scores, egg production (%), egg weights, eggshell weights, eggshell thickness, total eggs laid and feed conversion per eggs laid.

Results

Body Weight (Table 5). When the supplement was provided to commercial-type egg laying hens, body weight was improved at Day 28 (p<0.05), Day 56 (p<0.05), Day 84 (p<0.05), Day 112 and Day 140 (p<0.05).

TABLE 5

Body Weight (grams)

| Trial Day | Control | CT |
|---|---|---|
| 0 | 1374.700 ^{a} | 1374.683 ^{a} |
| 28 | 1514.700 ^{a} | 1563.833 ^{b} |
| 56 | 1562.317 ^{a} | 1645.100 ^{b} |
| 84 | 1580.633 ^{a} | 1679.767 ^{b} |
| 112 | 1624.967 ^{a} | 1693.383 ^{a} |
| 140 | 1657.633 ^{a} | 1742.500 ^{b} |

Note:
rows without a common superscript are significantly different (P < 0.05) as determined by Least Significant Difference.

Bird Health (Table 6). All remaining hens at the end of the study were gross necropsied and intestinal lesion scores were determined and fat pad as % of body weight was measured. Fat pad improved (p<0.05) and intestinal lesion scores were reduced and improved by supplementation.

TABLE 6

Day 140 of Trial

| Bird Health | Control | CT |
|---|---|---|
| Fat Pad (% of body weight) | 2.656 ^{a} | 2.852 ^{b} |
| Intestinal lesion scores | 0.917 ^{a} | 0.467 ^{b} |

Note:
rows without a common superscript are significantly different (P < 0.05) as determined by Least Significant Difference.

Egg Production (Table 7). Egg production (%) is measured as a percentage of eggs produced per hen per treatment group per day. Egg production (%) was increased in periods Days 0-28 (p<0.05), Days 29-56 (p<0.05), Days 57-84 (p<0.05), Days 85-112 (p<0.05), Days 113-140 (p<0.05) and for the duration of the trial period of Days 0-140 (p<0.05) when hen's feed was supplemented.

TABLE 7

Egg Production (%)

| Trial Period | Control | CT |
|---|---|---|
| Days 0-28 | 44.762% ^{a} | 52.976% ^{a} |
| Days 29-56 | 84.286% ^{a} | 89.643% ^{b} |
| Days 57-84 | 87.560% ^{a} | 96.012% ^{b} |
| Days 85-112 | 84.643% ^{a} | 97.619% ^{b} |
| Days 113-140 | 86.964% ^{a} | 90.060% ^{b} |
| Days 0-140 | 77.643% ^{a} | 85.262% ^{b} |

Note:
rows without a common superscript are significantly different (P < 0.05) as determined by Least Significant Difference.

Egg Weights (Table 8). Egg weights were measured in grams per egg average per treatment group. Average egg weights improved in measurement periods Days 0-28 (p<0.05), Days 29-56, Days 57-84, Days 85-112, and Days 113-140 from hens diets with the supplement.

TABLE 8

Average Egg Weights (grams)

| Trial Period | Control | CT |
|---|---|---|
| Days 0-28 | 43.975 ^{a} | 49.858 ^{b} |
| Days 29-56 | 54.150 ^{a} | 60.008 ^{a} |
| Days 57-84 | 62.588 ^{a} | 69.833 ^{a} |
| Days 85-112 | 62.583 ^{a} | 70.133 ^{a} |
| Days 113-140 | 62.500 ^{a} | 69.938 ^{a} |

Note:
rows without a common superscript are significantly different (P < 0.05) as determined by Least Significant Difference.

Egg Shell Weights (Table 9). Egg weights were measured in grams per egg average per treatment group. Average eggshell weights improved in measurement periods Days 29-56, Days 57-84, Days 85-112, and Days 113-140.

TABLE 9

Average Egg Shell Weights (grams)

| Trial Period | Control | CT |
|---|---|---|
| Days 0-28 | 6.257 ^{a} | 6.215 ^{a} |
| Days 29-56 | 6.722 ^{a} | 7.188 ^{a} |
| Days 57-84 | 6.704 ^{a} | 7.233 ^{a} |
| Days 85-112 | 6.683 ^{a} | 7.279 ^{a} |
| Days 113-140 | 6.729 ^{a} | 7.198 ^{a} |

Note:
rows without a common superscript are significantly different (P < 0.05) as determined by Least Significant Difference.

Total Eggs Laid, Total Kilograms of Eggs Laid (Table 10). Total eggs laid and total weight (kilograms) of eggs laid by hens fed the supplement improved.

TABLE 10

Days 0-140 of Trial

| Bird Health | Control | CT |
|---|---|---|
| Number of Eggs Laid | 326 ^{a} | 358 ^{b} |
| Total kg of eggs laid | 18.640 ^{a} | 22.902 ^{b} |

Note:
rows without a common superscript are significantly different (P < 0.05) as determined by Least Significant Difference.

Feed conversion (kilogram feed per dozen eggs) (Table 11). Feed conversion of kilograms per dozen of eggs is calculated by taking the total amount of feed consumed per treatment group divided by the total number of dozens of eggs produced by each treatment group. A lower number means that a greater number of eggs are produced assuming an equal amount of feed consumed (or, said another way, an equal number of eggs could be produced from a hen consuming less feed). Hens fed the supplement improved feed conversion (kg feed per dozen of eggs) in trial periods: Days 0-28 (p<0.05), Days 29-56 (p<0.05), Days 57-84 (p<0.05), Days 85-112 (p<0.05), Days 113-140 (p<0.05) and overall Days 0-140 (p<0.05).

TABLE 11

Feed Conversion
(kg feed per dozen eggs)

| Trial Period | Control | CT |
|---|---|---|
| Days 0-28 | 2.697 ^{a} | 2.274 ^{b} |
| Days 29-56 | 1.527 ^{a} | 1.428 ^{b} |
| Days 57-84 | 1.499 ^{a} | 1.359 ^{b} |
| Days 85-112 | 1.584 ^{a} | 1.367 ^{b} |
| Days 113-140 | 1.572 ^{a} | 1.487 ^{b} |
| Days 0-140 | 1.986 ^{a} | 1.797 ^{b} |

Note:
rows without a common superscript are significantly different (P < 0.05) as determined by Least Significant Difference.

Feed conversion (kilogram feed per kilogram of eggs) (Table 12). Feed conversion of kilograms of feed per kilograms of eggs produced is calculated by taking the total amount of feed consumed per treatment group (in kilograms) divided by the total number of kilograms of eggs produced by each treatment group. A lower number means that a greater total weight of eggs are produced assuming an equal amount of feed consumed (or, said another way, an equal weight of eggs could be produced from a hen consuming less feed). Egg laying hens fed the supplement improved feed conversion (kg feed per kg of eggs) in trial periods: Days 0-28 (p<0.05), Days 29-56 (p<0.05), Days 57-84 (p<0.05), Days 85-112 (p<0.05), Days 113-140 (p<0.05) and overall Days 0-140 (p<0.05).

TABLE 12

Feed Conversion
(kg feed per kg eggs)

| Trial Period | Control | CT |
|---|---|---|
| Days 0-28 | 5.111 ^{a} | 3.802 ^{b} |
| Days 29-56 | 2.350 ^{a} | 1.984 ^{b} |
| Days 57-84 | 1.996 ^{a} | 1.622 ^{b} |
| Days 85-112 | 2.109 ^{a} | 1.624 ^{b} |
| Days 113-140 | 2.096 ^{a} | 1.772 ^{b} |
| Days 0-140 | 2.895 ^{a} | 2.341 ^{b} |

Note:
rows without a common superscript are significantly different (P < 0.05) as determined by Least Significant Difference.

The complete disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. If any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A supplement comprising free amino acids L-glutamine or a salt thereof, L-leucine or a salt thereof, and L-arginine or a salt thereof, and active IGF-1, wherein the L-glutamine or salt thereof, L-leucine or salt thereof, and L-arginine or salt thereof are present at a ratio of 2-4:1:1, 3:0.5-2:1, or 3:1:0.5-3.

2. The supplement of claim 1 wherein the at least one of the L-glutamine, the L-leucine, and the L-arginine is in a salt form.

3. The supplement of claim 1 wherein the amount of L-glutamine or a salt thereof is at least 0.5 grams to no greater than 500 grams amino acid/kilogram supplement.

4. The supplement of claim 1 wherein the amount of L-leucine or a salt thereof is at least 0.1 grams to no greater than 100 grams amino acid/kilogram supplement.

5. The supplement of claim 1 wherein the amount of L-arginine or a salt thereof is at least 0.1 grams to no greater than 200 grams amino acid/kilogram supplement.

6. The supplement of claim 1 wherein the amount of active IGF-1 is at least 100 micrograms to no greater than 2000 micrograms active IGF-1/kilogram supplement.

7. The supplement of claim 1 wherein the amount of L-glutamine or a salt thereof is at least 0.5 grams to no greater than 500 grams amino acid/kilogram supplement, the amount of L-leucine or a salt thereof is at least 0.1 grams to no greater than 100 grams amino acid/kilogram supplement, the amount of L-arginine or a salt thereof is at least 0.1 grams to no greater than 200 grams amino acid/kilogram supplement, and the amount of active IGF-1 is at least 100 grams to no greater than 2000 microgram IGF-1/kilogram supplement.

8. The supplement of claim 1 wherein the active IGF-1 is recombinant IGF-1.

9. The supplement of claim 1 further comprising a food product.

10. A food product comprising the supplement of claim 1 present in the food product at an amount of at least 0.000005 g/ton and no greater than 0.005 g/ton, or at an amount of at least 50 grams/ton (g/ton) of the food product and no greater than 2600 g/ton.

11. (canceled)

12. The food product of claim 10 wherein the total weight percent (wt %) of the L-glutamine or salt thereof, the L-leucine or salt thereof, and the L-arginine or salt thereof is less than the total wt % of other amino acids in the supplement.

13. The food product of claim 10 wherein one, two, or all three of the L-glutamine, the L-leucine, and the L-arginine is in a salt form.

14. The food product of claim 10 wherein the amount of L-glutamine or a salt thereof is at least 0.0000002 wt %.

15. The food product of claim 10 wherein the amount of L-leucine or a salt thereof is at least 0.00000006 wt %.

16. The food product of claim 10 wherein the amount of L-arginine or a salt thereof is at least 0.00000006 wt %.

17. A method comprising administering to an animal an effective amount of (i) a food product comprising the supplement of claim 1.

18-20. (canceled)

21. The method of claim 17 wherein the food product comprises the supplement at an amount of at least 50 g/ton.

22. The method of claim 17 wherein the animal is a porcine animal or an avian animal.

23-29. (canceled)

30. A method of increasing body weight, improving health as measured by reduced intestinal lesions and/or increased fat pad as a percentage of body weight, increasing egg production, increasing total eggs laid, and/or improving feed conversion in an avian layer, comprising administering to the animal a composition supplemented with the composition of claim 1, wherein the increasing body weight, improving health as measured by reduced intestinal lesions and/or increased fat pad as a percentage of body weight, increasing egg production, increasing total eggs laid, and/or improving feed conversion is a change compared to animals in a similar environment but not fed the food product containing the supplement.

31. The method of claim 30 wherein the avian layer is a chicken.

32-35. (canceled)
